(19) United States
(12) Patent Application Publication
(10) Pub. No.: US 2006/0178415 A1
(43) Pub. Date: Aug. 10, 2006

(54) Azole derivatives as antifungal agents

(76) Inventors: Mohammad Salman, Gurgaon (IN); Jitendra Anant Sattigeri, Gurgaon (IN); Rita Latoch, Chandigarh (IN); Sachin Sethi, Gurgaon (IN); Ashok Rattan, New Delhi (IN)

Correspondence Address: RANBAXY INC., 600 COLLEGE ROAD EAST, SUITE 2100, PRINCETON, NJ, 08540, US

(21) Appl. No.: 10/525,438

(22) PCT Filed: Sep. 12, 2002
(86) PCT No.: PCT/IB02/03740

§ 371 Date: Feb. 13, 2006

(30) Foreign Application Priority Data
Aug. 26, 2002 (IB) 02/03435

(51) Int. Cl. A61K 31/4196; A61K 31/4172

(52) U.S. Cl. 514/384; 514/383; 514/400; 548/335.5; 548/263.2; 548/267.2

(57) ABSTRACT

The present invention relates to novel azole derivatives of Formula I, as potential antifungal agents. This invention also relates to pharmaceutical compositions containing the compounds of the present invention and their use in treating and/or preventing the fungal infections in mammals, preferably humans.

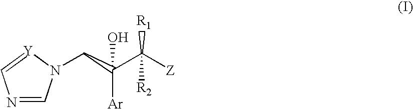

### FIELD OF THE INVENTION

The present invention relates to novel azole derivatives of Formula I, as potential antifungal agents.

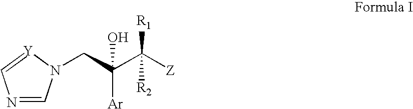

This invention also relates to pharmaceutical compositions containing the compounds of the present invention and their use in treating and/or preventing the fungal infections in mammals, preferably humans.

### BACKGROUND OF THE INVENTION

Life threatening, systemic fungal infections continue to be a significant problem in health care today. In particular, patients who become “immunocompromised” as a result of diabetes, cancer, prolonged steroid therapy, organ transplantation anti-rejection therapy, the acquired immune deficiency syndrome, (AIDS) or other physiologically or immunologically compromising syndromes are especially susceptible to opportunistic fungal infections.

Since the 1950's and until recently, the key opportunistic fungal pathogens were Candida albicans, Aspergillus fumigatus, and Zygomycetes, which cause mucormycosis, a rapidly fatal infection especially in diabetic patients. Today, non-albicans Candida isolates have become more frequent, as have other Aspergillus species. Candida species are now the fourth most common cause of nosocomial blood stream infection and they are associated with an extremely high mortality rate of 40%. From 1980 to 1990, the incidence of fungal infections in the US hospitals nearly doubled, from approximately 2 to 3.85 per 1000 patient days. The most marked increase in fungal infection rates occurred not only in transplant units or oncology centres, but also in surgical services. These changing patterns demonstrate that fungal infections are no longer limited to the most severely immunocompromised patients.

During the past two decades, a substantial shift in the epidemiology of candidemia due to different Candida species has occurred. In the 1960's and 1970's Candida albicans accounted for 85-90% of cases of candidemia. In 1999 however, only 42% of candidemia cases were caused by C.albicans, while non-albicans Candida accounted for the remainder.

Cryptococosis is a leading cause of morbidity among the AIDS patients. The incidence of life threatening cryptococcal infection among these patients have been estimated to vary from 10 to 30%; 10-20% of the patients die during initial therapy and 30 to 60% patients succumb within a year. Penicillinium mameffei has been frequently isolated from HIV positive patients, especially in Southeast Asia.

The most common causative agent of mucormycosis is Rhizopus, a common bread mould that lives on any organic material. Other pathogens include Mucor, Rhizomucor and Absidia. Zygomycetes include twenty different fungi, all appearing the same histologically. The severely immunocompromised patient may become infected with Zygomycetes via respiratory inhalation.

Fusarium is the most prevalent plant fungus worldwide, and it is now recognised as human pathogen as well. Fusarium infections can occur in immunocompetent or immunosuppressed individuals. Fusarium infection is life threatening and associated with a poor prognosis.

Penicillium marneffei is an environmental fungi that can cause serious life threatening infections in immunosuppressed patients. Penicillium marneffei has gained particular attention during the AIDS pandemic, as it may produce disease that is clinically indistinguishable from disseminated histoplasmosis.

Invasive aspergillosis has become a leading cause of death, mainly among patients suffering from acute leukaemia or after allogenic bone marrow transplant and after cytotoxic treatment of these conditions. It also occurs in patients with condition such as AIDS and chronic granulomatous disease. At present, only Amphotericin B and itraconazole are available for treatment of aspergillosis. In spite of their activity in-vitro, the effect of these drugs in-vivo against Aspergillus fumigatus remains low and as a consequence mortality from invasive aspergillosis remains high.

Although the first agent with antifungal activity, Griseofulvin was isolated in 1939 and the first azole and polyene antifungal agents were reported in 1944 and 1949, respectively (Clin. Microbiol. Rev., 1988; 1:187), it was not until 1960 that Amphotericin B (I. J. Am. Acad, Dermatol, 1994; 31:S51), which is still the “gold standard” for the treatment of severe systemic mycoses, was introduced (Antimicrob. Agents Chemother., 1996; 40:279). Despite the general effectiveness of Amphotericin B, it is associated with a number of complications and unique toxicities that limit its use. Furthermore, the drug is poorly absorbed from the gastrointestinal tract necessitating intravenous administration and also penetrates poorly into the cerebrospinal fluid (CSF) of both normal and inflamed meninges. The problems associated with Amphotericin B stimulated search for newer agents.

By 1980, members of the four major classes of antifungal agents, viz. polyenes, azoles, morpholines and allylamines had been identified. And advances made during the 1990's led to the addition of some new classes such as the Candins, and the Nikkomycins (Exp. Opin. Investig. Drugs, 1997; 6:129). However, with 15 different marketed drugs worldwide, (Drugs, 1997; 53:549) the azoles are currently the most widely used and studied class of antifungal agents.

Azole antifungal agents prevent the synthesis of ergosterol, a major component of fungal plasma membranes, by inhibiting the cytochrome P450 dependent enzyme lanosterol demethylase (referred to as 14-α-sterol demethylase or P450_{DM}). This enzyme also plays an important role in the cholesterol synthesis in mammals. When azoles are present in therapeutic concentrations, their antifungal efficacy is attributed to their greater affinity for fungal P450_{DM} than for the mammalian enzyme (Curr. Opin. Chem. Biol., 1997; 1:176).

The azole antifungals currently in clinical use contain either two or three nitrogens in the azole ring and are thereby classified as imidazoles (e.g. ketoconazole, miconazole and clotrimazole) or triazoles (e.g. itraconazole and fluconazole), respectively. With the exception of Ketoconazole, use of the imidazoles is limited to the treatment of superficial mycoses, whereas the triazoles have a broad range of applications in the treatment of both superficial and systemic fungal infections. Another advantage of the triazoles is their greater affinity for fungal rather than mammalian cytochrome P-450 enzymes.

The use of Ketoconazole is severely restricted partly due to its poor toxicity and pharmacokinetic profile and also the fact that none of the opportunistic fungal infections like aspergillosis, candidemia and cryptococcosis are responsive to it (Antifungal Agents, pgs 401-410 In. G. L. Mandel, J. E. Bennett and R. Dolin (ed.) Principles and practice of infectious diseases, 4^{th} ed. Churchill Livingstone, Inc. New York, N.Y.). Fluconazole is the current drug of is choice for treatment of infectious caused by Candida species and C. neoformans. However, management of serious infectious due to Candida species are becoming increasingly problematic because of rising incidence of non-albicans species and the emergence non-albicans isolates resistant to both amphotericin B and the newer azoles. (Am. J. Med., 1996; 100:617). Also, fluconazole's spectrum suffers because it has only weak inhibitory activity against isolates of Aspergillus species. With regard to the prevention of invasive aspergillosis, a number of antifungal regimens have been suggested for neutropenic patients but only itraconazole has been considered for primary prophylaxis. However, its activity in the clinic remains mixed as it shows variable oral availability, low solubility and very high protein binding besides causing ovarian cancer in animals.

Voriconazole, the fluconazole analog launched recently by Pfizer exhibits 1.6 and 160 fold greater inhibition of ergosterol P⁴⁵⁰_{DM} in C. albicans and A. fumigatus lysates respectively, compared to fluconazole (Clin. Microbiol. Rev., 1999; 12:40). Voriconazole was designed to retain the parenteral and oral formulation advantage of fluconazole while extending its spectrum to moulds, insufficiently treated yeasts and less common fungal pathogens. But though oral bioavailability of voriconazole is high, there is saturable metabolism which results in a more than proportional increase in exposure with increased oral and I.V. doses. Inter-individual variability in voriconazole pharmacokinetics is high and concerns about its occular toxicity potentials remain to be resolved.

The development of some of the earlier compounds which included SCH 39304 (Genoconazole), TAK-1 87, SCH-42427 (Saperconazole), BAY R-8783 (Electrazole) and D-0870 had to be discontinued as a result of safety concerns.

ER-30346 (Ravuconazole), the fluconazole analog under development shows anti-aspergillus profile, at best only equal to that of itraconazole. Schering Plough compound SCH 56592 (Posaconazole) shows potent broad spectrum activity against primary opportunistic fungal pathogens including Candida spp., C. neoformans and Aspergillus spp. However, it has a pharmacokinetic profile similar to that of itraconazole and is not detectable in CSF, even when the serum drug concentration after several days of treatment are 25 to 100 times above the MIC for the most resistant C. neoformans. (Antimicrobial Agents and Chemother, 1996; 40:1910, 36^{th} interscience Conference on Antimicrobial agents and chemotherapy, September 1996, New Orleans Abst. Drugs of the Future, 1996;, 21:20).

Thus, the antifungals in the market, as well as under development suffer with drawbacks such as toxicity, narrow spectrum of activity and fungistatic profile rather than fungicidal. Some of them also exhibit drug-drug interactions and as a result, therapy becomes complex. In view of the high incidence of fungal infections in immunocompromised patients and the recent trends for the steady increase of the population of such patients, demands for new antifungal agents with broad spectrum of activity and good pharmacokinetic properties has increased. Therefore, development of antifungal agents is still a big challenge.

### SUMMARY OF THE INVENTION

The present invention provides novel compounds of Formula I: 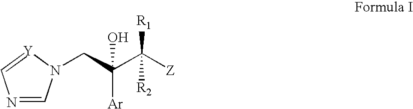
and its pharmaceutically acceptable salts, esters, enantiomers, diastereomers, N-oxides, prodrugs, metabolites, polymorphs, pharmaceutically acceptable solvates,
wherein
Ar is phenyl or a substituted phenyl having one to three substituents independently selected from halogen (chlorine, fluorine, bromine, iodine), nitro, cyano, lower (C₁₋₄)alkyl, lower(C₁₋₄)alkoxy, perhalo lower(C₁₋₄)alkyl or perhalo lower(C₁₋₄)alkoxy; five to seven membered heterocyclic ring containing one to four heteroatoms selected from the group consisting of oxygen, nitrogen and sulphur, the more preferred Ar is 2,4-difluorophenyl;
R₁ and R₂ are independently selected from the group consisting of hydrogen, straight chain or branched alkyl groups having 1 to 3 carbon atoms for example methyl, ethyl, propyl or isopropyl and their combinations thereof; the preferred alkyls are methyl and ethyl ; the more preferred combination is when R₁ is methyl and R₂ is hydrogen;
Y is CH or N;
Z is selected from the group consisting of 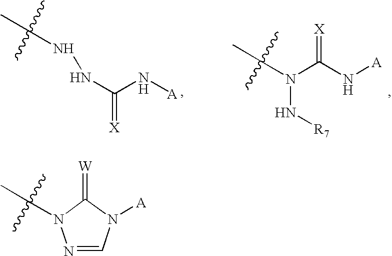
   wherein
X is selected from S, O, CH—NO₂, and N—CN;
W is selected from S, CH—NO₂, and N—CN;
A is hydrogen, unsubstituted or substituted lower (C₁₋₁₀)alkyl, said substituents being halogen (fluorine, chlorine, bromine or iodine), hydroxy, lower (C₁₋₄)alkoxy, lower (C₁₋₄)perhaloalkyl, lower (C₁₋₄)perhaloalkoxy; optionally substituted naphthyl; unsubstituted or substituted aromatic or non aromatic 5-6 membered rings with or without one to four heteroatoms independently selected from the group consisting of oxygen, nitrogen and sulphur, said substituents independently selected from one or more groups such as halogen (fluorine, chlorine, bromine or iodine), nitro, cyano, hydroxy, lower(C₁₋₄)alkyl, lower(C₁₋₄)alkoxy, lower (C₁₋₄)perhaloalkyl, lower (C₁₋₄)perhaloalkoxy, BR₃, substituted or unsubstituted five or six membered heterocyclic ring systems containing one to four heteroatoms selected from the group consisting of oxygen, nitrogen and sulphur, said heterocycylic substituents being (C_{1-C}₈)alkanoyl, lower (C₁-C₄)alkyl, lower (C₁-C₄)alkoxy carbonyl, N lower (C₁-C₄)alkylaminocarbonyl, N,N-dilower (C₁-C₄)alkylaminocarbonyl, N-lower (C₁-C₄)alkylaminothiocarbonyl, N,N-di(lower alkyl)(C₁-C₄)aminothiocarbonyl, N-lower (C₁-C₄)alkyl sulphonyl, phenyl substituted lower (C₁-C₄)alkyl sulphonyl, N-lower (C₁-C₄)alkyl amino, N,N-di(lower alkyl)(C₁-C₄)amino,unsubstituted or substituted phenyl, said substituents being halogen (fluorine, chlorine, bromine or iodine), hydroxy, lower (C₁₋₄)alkoxy, lower (C₁₋₄)perhaloalkyl, lower (C₁₋₄)perhaloalkoxy, nitro, cyano, amino, N(R₄)₂, 5-6 membered heterocyclic rings, the preferred heterocycles being 1,3-imidazolyl; 1,2,4 triazolyl ; —CHR₅R₆;
wherein
R₃ is a five or six membered aromatic or non aromatic ring with or without heteroatoms (oxygen, nitrogen and sulphur);
B is independently selected from (CH₂)ₘ, —O(CH₂)ₘ, —S(CH₂)ₘ;
m is an integer from 1 to 4;
R₄ is hydrogen, unsubstituted or substituted lower (C₁₋₄)alkyl;
R₅ is —COQ, where Q=OR₄, —N(R₄)₂;
R₆ is independently selected from hydrogen, straight chain or branched alkyl with or without substituents, said substituents being halogen (fluorine, chlorine, bromine or iodine), hydroxy, lower (C₁₋₄)alkyl, lower (C₁₋₄)alkoxy, lower (₁₋₄)perhaloalkyl, lower (C₁₋₄)perhaloalkoxy, SR₄; phenyl or phenyl substituted with halogen (fluorine, chlorine, bromine or iodine), hydroxy, lower (C₁₋₄)alkoxy, lower (C₁₋₄)perhaloalkyl, lower (C₁₋₄)perhaloalkoxy, SR₄; heterocyclic rings or substituted heterocyclic rings with heteroatoms selected from oxygen, nitrogen and sulphur, substituents on heterocyclic rings are independently selected from halogen (fluorine, chlorine, bromine or iodine), hydroxy, lower (C₁₋₄)alkyl, lower (C₁₋₄)alkoxy, lower (C₁₋₄)perhaloalkyl, lower (C₁₋₄)perhaloalkoxy, SR₄; the preferred heterocyclic rings are imidazole and indole;
R₇ is H or selected from the group consisting of 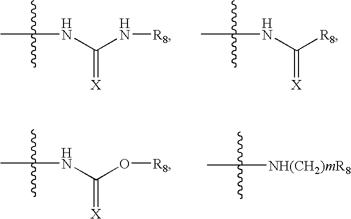
   wherein
R₈ is independently selected from hydrogen, unsubstituted or substituted lower (C₁₋₄)alkyl, aralkyl, aromatic or non aromatic 5-6 membered rings with or without one to four heteroatoms selected independently from the group consisting of oxygen, nitrogen and sulphur.

The present invention also provides pharmaceutical compositions for the treatment of fungal infections. These compositions comprise an effective amount of at east one of the above compounds of Formula I and/or an effective amount of at least one physiologically acceptable acid addition salts thereof with a pharmaceutically acceptable carriers.

The compound represented by the Formula I may be used as a salt thereof, examples of such salts are pharmacologically acceptable salts such as inorganic acid salts (e.g. hydrochloride, hydrobromide, sulphate, nitrate and phosphate), organic acid salts(e.g. acetate, tartarate, citrate, fumarate, maleate, tolounesulphonate and methanesulphonate). When carboxyl group is included in the Formula I as a substituent, it may be an alkali metal salt (e.g. sodium, potassium, calcium, magnesium, and the like).

The present invention also includes within its scope prodrugs of the compounds of Formula I. In general, such prodrugs will be functional derivatives of these compounds which are readily converted in vivo into defined compounds. Conventional procedures for the selection and preparation of suitable prodrugs are known.

The compounds represented by the Formula I, or a salt thereof, have two or more stereoisomers due to the presence of one or more asymmetric carbon atom(s) in their molecule. It should be understood that any of such stereoisomers as well as a mixture thereof is within the scope of the present invention.

The invention also includes polymorphs and pharmaceutically acceptable solvates of these compounds, as well as metabolites. This invention further includes pharmaceutical compositions comprising the compounds of Formula I, their prodrugs, metabolites, enantiomers, diastereomers, N-oxides, polymorphs, solvates or pharmaceutically acceptable salts thereof, in combination with a pharmaceutically acceptable carrier and optionally included excipients.

The illustrative list of particular compounds of the invention is given below and are also shown in Tables I and II:
1-t-Butoxycarbonyl-2-[((1R,2R)-2-(2,4-difluorophenyl)-2-hydroxy-1-methyl-3-(1H-1,2,4-triazol-1-yl)propyl]-4-[4-fluorophenyl]thiosemicarbazide (Compound No. 1)
1-t-Butoxycarbonyl-2-[(1R,2R)-2-(2,4-difluorophenyl)-2-hydroxy-1-methyl-3-(1H-1,2,4-triazol-1-yl)propyl]-4-[2,4-difluorophenyl]thiosemicarbazide (Compound No. 2)
1-t-Butoxycarbonyl-2-[(1R,2R)-2-(2,4-difluorophenyl)-2-hydroxy-1-methyl-3-(1H-1,2,4-triazol-1-yl)propyl]-4-[4-trifluoromethylphenyl]thiosemicarbazide (Compound No. 3)
1-t-Butoxycarbonyl-2-[(1R,2R)-2-(2,4-difluorophenyl)-2-hydroxy-1-methyl-3-(1H-1,2,4-triazol-1-yl)propyl]-4-[2,4-dimethoxyphenyl]thiosemicarbazide (Compound No. 4)
1-t-Butoxycarbonyl-2-[(1R,2R)-2-(2,4-difluorophenyl)-2-hydroxy-1-methyl-3-(1H-1,2,4-triazol-1-yl)propyl]-4-[4-(tetrahydropyranyloxy)phenyl]thiosemicarbazide (Compound No. 5)
1-t-Butoxycarbonyl-2-[(1R,2R)-2-(2,4-difluorophenyl)-2-hydroxy-1-methyl-3-(1H-1,2,4-triazol-1-yl)propyl]-4-[4-trifluoromethoxyphenyl]thiosemicarbazide (Compound No. 6)
1-t-Butoxycarbonyl-2-[(1R,2R)-2-(2,4-difluorophenyl)-2-hydroxy-1-methyl-3-(1H-1,2,4-triazol-1-yl)propyl]-4-[4-(2,2,3,3-tetrafluoropropoxy)phenyl]thiosemicarbazide (Compound No. 7)
1-t-Butoxycarbonyl-2-[(1R,2R)-2-(2,4difluorophenyl)-2-hydroxy-1-methyl-3-(1H-1,2,4-triazol-1-yl)propyl]-4-[4-nitrophenyl]thiosemicarbazide (Compound No. 8)
1-t-Butoxycarbonyl-2-[(1R,2R)-2-(2,4-difluorophenyl)-2-hydroxy-1-methyl-3-(1H-1,2,4-triazol-1-yl)propyl]-4-[4-([1,2,3,4-tetrazol-1-yl])phenyl]thiosemicarbazide (Compound No. 9)
1-t-Butoxycarbonyl-2-[(1R,2R)-2-(2,4-difluorophenyl)-2-hydroxy-1-methyl-3-(1H-1,2,4-triazol-1-yl)propyl]-4-[4-(1,2,3,4-tetrazol-2-yl)phenyl]thiosemicarbazide (Compound No. 10)
1-t-Butoxycarbonyl-2-[(1R,2R)-2-(2,4difluorophenyl)-2-hydroxy-1-methyl-3-(1H-1,2,4-triazol-1-yl)propyl]-4-[4-cyanophenyl]thiosemicarbazide (Compound No. 11)
1-t-Butoxycarbonyl-2-[(1R,2R)-2-(2,4-difluorophenyl)-2-hydroxy-1-methyl-3-(1H-1,2,4-triazol-1-yl)propyl]-4-[4-[4-chlorophenyl]piperizin-1-yl]phenyl]thiosemicarbazide (Compound No. 12)
1-t-Butoxycarbonyl-2-[(1R,2R)-2-(2,4-difluorophenyl)-2-hydroxy-1-methyl-3-(1H-1,2,4-triazol-1-yl)propyl]-4-[4-(N,N-dimethylamino)phenyl]thiosemi-carbazide (Compound No. 13)
1-t-Butoxycarbonyl-2-(1R,2R)-2-(2,4-difluorophenyl)-2-hydroxy-1-methyl-3-(1H-1,2,4-triazol-1-yl)propyl]-4-napth-1-yl thiosemicarbazide (Compound No. 14)
1-t-Butoxycarbonyl-2-(1R,2R)-2-(2,4-difluorophenyl)-2-hydroxy-1-methyl-3-(1H-1,2,4-triazol-1-yl)propyl]-4-octylthiosemicarbazide (Compound No. 15)
1-t-Butoxycarbonyl-2-[(1R,2R)-2-(2,4-difluorophenyl)-2-hydroxy-1-methyl-3-(1H-1,2,4-triazol-1-yl)propyl]-4-t-butyl thiosemicarbazide (Compound No.16)
Methyl-2-[1-t-butoxycarbonyl-2-[(1R,2R)-2-(2,4-difluorophenyl)-2-hydroxy-1-methyl-3-(1H-1,2,4-triazol-1-yl)propyl]thiosemicarbazid-4-yl]acetate (Compound No. 17)
Methyl-2-phenyl-2-[1-t-butoxycarbonyl-2-[(1R,2R)-2-(2,4-difluorophenyl)-2-hydroxy-1-methyl-3-(1H-1,2,4-triazol-1-yl)propyl]thiosemicarbazid-4-yl]acetate (Compound No. 18)
Methyl-2-[t-butyldimethylsilyloxymethyl]-2-[1-t-butoxycarbonyl-2-[(1R,2R)-2-(2,4-difluorophenyl)-2-hydroxy-1-methyl-3-(1H-1,2,4-triazol-1-yl)propyl]thiosemicarbazid-4-yl]acetate (Compound No. 19)
Methyl-2-[methylthioethyl]-2-[1-t-butoxycarbonyl-2-[(1R)-2R)-2-(2,4-difluorophenyl)-2-hydroxy-1-methyl-3-(1H-1,2,4-triazol-1-yl)propyl]thiosemicarbazid-4-yl]acetate(Compound No. 20)
Methyl-2-benzyl-2-[1-t-butoxycarbonyl-2-[(1R,2R)-2-(2,4-difluorophenyl)-2-hydroxy-1-methyl-3-(1H-1,2,4-triazol-1-yl)propyl]thiosemicarbazid-4-yl]acetate (Compound No. 21)
Methyl-2-isobutyl-2-[1-t-butoxycarbonyl-2-[(1R,2R)-2-(2,4-difluorophenyl)-2-hydroxy-1-methyl-3-(1H-1,2,4-triazol-1-yl)propyl]thiosemicarbazid-4-yl]acetate (Compound No. 22)
1-t-Butoxycarbonyl-2-[(1R,2R)-2-(2,4-difluorophenyl)-2-hydroxy-1-methyl-3-(1H-1,2,4-triazol-1-yl)propyl]-4-[2-furanmethyl]thiosemicarbazide (Compound No. 23)
1-t-Butoxycarbonyl-2-[(1R,2)-2-(2,4-difluorophenyl)-2-hydroxy-1-methyl-3-(1H-1,2,4-triazol-1-yl)propyl]-4-[2-thiophenmethyl]thiosemicarbazide (Compound No. 24)
1-t-Butoxycarbonyl-2-[(1R,2R)-2-(2,4-difluorophenyl)-2-hydroxy-1-methyl-3-(1H-1,2,4-triazol-1-yl)propyl]-4-[4-chlorophenyl]semicarbazide (Compound No. 25)
1-t-Butoxycarbonyl-2-[(1R,2R)-2-(2,4-difluorophenyl)-2-hydroxy-1-methyl-3-(1H-1,2,4-triazol-1-yl)propyl]-4-[4(2,2,3,3-tetrafluoropropoxy)phenyl]semicarbazide (Compound No. 26)
1-t-Butoxycarbonyl-2-[(1R,2R)-2-(2,4-difluorophenyl)-2-hydroxy-1-methyl-3-(1H-1,2,4-triazol-1-yl)propyl]-4-[2,4-dimethoxyphenyl]semicarbazide (Compound No. 27)
2-[(1R,2R)-2-(2,4-difluorophenyl)-2-hydroxy-1-methyl-3-(1H-1,2,4-triazol-1-yl)propyl]-4-[4-chlorophenyl]semicarbazide (Compound No. 28)
2-[(1R,2R)-2-(2,4-difluorophenyl)-2-hydroxy-1-methyl-3-(1H-1,2,4-triazol-1-yl)propyl]-4-phenyl thiosemicarbazide (Compound No. 29)
2-[(1R,2R)-2-(2,4-difluorophenyl)-2-hydroxy-1-methyl-3-(1H-1,2,4-triazol-1-yl)propyl]-4-[4-hydroxyphenyl]thiosemicarbazide (Compound No. 30)
2-[(1R,2R)-2-(2,4-difluorophenyl)-2-hydroxy-1-methyl-3-(1H-1,2,4-triazol-1-yl)propyl]-4-[4-(2,2,3,3-tetrafluoropropoxy)phenyl]thiosemicarbazide (Compound No. 31)
2-[(1R,2R)-2-(2,4-difluorophenyl)-2-hydroxy-1-methyl-3-(1H-1,2,4-triazol-1-yl)propyl]-4-[2,4-dimethoxyphenyl]thiosemicarbazide (Compound No. 32)
2-[(1R,2R)-2-(2,4-difluorophenyl)-2-hydroxy-1-methyl-3-(1H-1,2,4-triazol-1-yl)propyl]-4-[4-trifluoromethylphenyl ]thiosemicarbazide (Compound No. 33)
2-[(1R,2R)-2-(2,4-Difluorophenyl)-2-hydroxy-1-methyl-3-(1H-1,2,4-triazol-1-yl)propyl]-4-[4-trifluoromethoxyphenyl]thiosemicarbazide (Compound No. 34)
2-[(1R,2R)-2-(2,4-difluorophenyl)-2-hydroxy-1-methyl-3-(1H-1,2,4-triazol-1-yl)propyl]-4-[2-furanmethyl]thiosemicarbazide (Compound No.35)
2-[(1R,2R)-2-(2,4-difluorophenyl)-2-hydroxy-1-methyl-3-(1H-1,2,4-triazol-1-yl)propyl]-4-[2-thiophenmethyl]thiosemicarbazide (Compound No.36)
2-[(1R,2R)-2-(2,4-Difluorophenyl)-2-hydroxy-1-methyl-3-(1H-1,2,4-triazol-1-yl)propyl]-4-[3-chloropyridin-6-yl]thiosemicarbazide (Compound No. 37)
2-[(1R,2R)-2-(2,4-Difluorophenyl)-2-hydroxy-1-methyl-3-(1H-1,2,4-triazol-1-yl)propyl]-4-[5chloro-3-tifluromethyl-pyridin-6-yl]thiosemicarbazide (Compound No. 38)
2-[(1R,2R)-2-(2,4-Difluorophenyl)-2-hydroxy-1-methyl-3-(1H-1,2,4-triazol-1-yl)propyl]-4-[quinolin-3-yl]thiosemicarbazide (Compound No. 39)
2-[(1R,2R)-2-(2,4-difluorophenyl)-2-hydroxy-1-methyl-3-(1,2,4-triazol-1-yl)propyl]-4-[4-(1,2,3,4-tetrazol-1-yl)phenyl]-(2H,4H)-1,2,4-triazol-3-thione (Compound No. 40)
2-[(1R,2R)-2-(2,4-difluorophenyl)-2-hydroxy-1-methyl-3-(1,2,4-triazol-1-yl)propyl]-4-[4-hydroxyphenyl]-(2H,4H)-1,2,4-triazol-3-thione (Compound No. 41)
2-[(1R,2R)-2-(2,4-difluorophenyl)-2-hydroxy-1-methyl-3-(1,2,4-triazol-1-yl)propyl]-4-[4-(2,2,3,3-tetrafluoropropoxy)phenyl]-(2H,4H)-1,2,4-triazol-3-thione (Compound No. 42)
2-[(1R,2R)-2-(2,4-difluorophenyl)-2-hydroxy-1-methyl-3-(1,2,4-triazol-1-yl)propyl]-4-[4-nitrophenyl]-(2H,4H)-1,2,4-triazol-3-thione (Compound No. 43)
2-[(1R,2R)-2-(2,4-difluorophenyl)-2-hydroxy-1-methyl-3-(1,2,4-triazol-1-yl)propyl]-4-[4-(1,2,3,4-tetrazol-2-yl))phenyl]-(2H,4H)-1,2,4-triazol-3-thione (Compound No. 44)
2-[(1R,2R)-2-(2,4-difluorophenyl)-2-hydroxy-1-methyl-3-(1,2,4-triazol-1-yl)propyl]-4-[4-trifluoromethylphenyl]-(2H,4H)-1,2,4-triazol-3-thione (Compound No. 45)
2-[(1R,2R)-2-(2,4-Difluorophenyl)-2-hydroxy-1-methyl-3-(1H-1,2,4-triazol-1-yl)propyl]-4-[4-trifluoromethoxyphenyl]-(2H,4H)-1,2,4-triazol-3-thione (Compound No. 46)
2-[(1R,2R)-2-(2,4-difluorophenyl)-2-hydroxy-1-methyl-3-(1,2,4-triazol-1-yl)propyl]-4-[4-cyanophenyl](2H,4H)-1,2,4-triazol-3-thione (Compound No. 47)
Methyl-2-[[(1R,2R)-2-(2,4-difluorophenyl)-2-hydroxy-1-methyl-3-(1H-1,2,4-triazol-1-yl)propyl]-(2H,4H)-1,2,4-triazol-3-thion-4-yl]acetate (Compound No. 48)
Methyl-2-hydroxymethyl-2-[[(1R,2R)-2-(2,4-difluorophenyl)-2-hydroxy-1-methyl-3-(1H-1,2,4-triaolyl)propyl]-(2H,4H)-1,2,4-triazol-3-thion-4-yl]acetate (Compound 1o No. 49)
Methyl-2-phenyl-2-[[(1R,2R)-2-2,4-difluorophenyl)-2-hydroxy-1-methyl-3-(1H-1,2,4-triaolyl)propyl]-(2H,4H)-1,2,4-triazol-3-thion-4-yl]acetate (Compound No. 50)
Methyl-2-isobutyl-2-[[(1R,2R)-2-(2,4-difluorophenyl)-2-hydroxy-1-methyl-3-(1H-1,2,4-triaolyl)propyl]-(2H,4H)-1,2,4-triazol-3-thion-4-yl]acetate (Compound No. 51)
Methyl-2-methylthioethyl-2-[[(1R,2R)-2-(2,4-difluorophenyl)-2-hydroxy-1-methyl-3-(1H-1,2,4-triaolyl)propyl]-(2H,4H)-1,2,4-triazol-3-thion-4-yl]acetate (Compound No. 52)
2-[(1R,2R)-2-(2,4-Difluorophenyl)-2-hydroxy-1-methyl-3-(1H-1,2,4-triazol-1-yl)propyl]-4-[2-furanmethyl]-(2H,4H)-1,2,4-triazol-3-thione (Compound No. 53)
2-[(1R,2R)-2-(2,4-Difluorophenyl)-2-hydroxy-1-methyl-3-(1H-1,2,4-triazol-1-yl)propyl]-4-[quinolin-3-yl]-(2H,4H)-1,2,4-triazol-3-thione (Compound No. 54)
2-[(1R,2R)-2-(2,4-Difluorophenyl)-2-hydroxy-1-methyl-3-(1H-1,2,4-triazol-1-yl)propyl]-4-[3-chloropyridin-6-yl]-(2H,4H)-1,2,4-triazol-3-thione (Compound No. 55).

2-[(1R,2R)-2-(2,4-Difluorophenyl)-2-hydroxy-1-methyl-3-(1H-1,2,4-triazol-1-yl)propyl]-4-[5-chloro-3-trifluoromethylpyridin-6-yl]-(2H,4H)-1,2,4-triazol-3-thione (Compound No. 56)

TABLE I

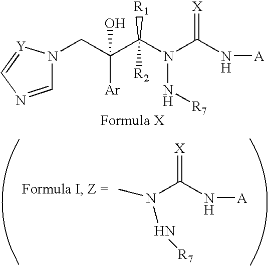

| Compound No. | Y | Ar | R₁ | R₂ | R₇ | X | A |
|---|---|---|---|---|---|---|---|
| 1 | N | 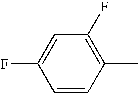 | CH₃ | H | BOC | S | 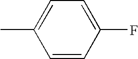 |
| 2 | N | 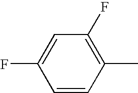 | CH₃ | H | BOC | S | 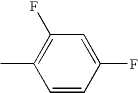 |
| 3 | N | 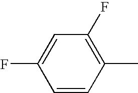 | CH₃ | H | BOC | S | 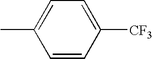 |
| 4 | N | 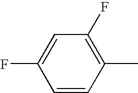 | CH₃ | H | BOC | S | 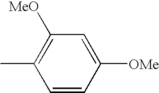 |
| 5 | N | 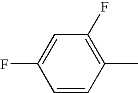 | CH₃ | H | BOC | S | 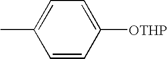 |
| 6 | N | 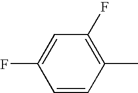 | CH₃ | H | BOC | S | 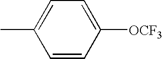 |
| 7 | N | 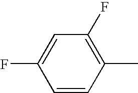 | CH₃ | H | BOC | S | 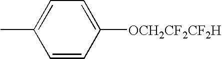 |
| 8 | N | 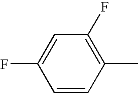 | CH₃ | H | BOC | S | 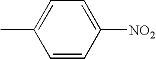 |
| 9 | N | 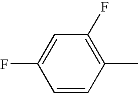 | CH₃ | H | BOC | S | 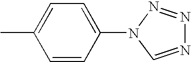 |
| 10 | N | 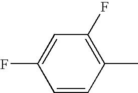 | CH₃ | H | BOC | S | 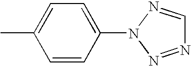 |
| 11 | N | 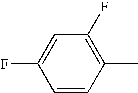 | CH₃ | H | BOC | S | 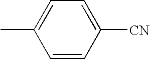 |
| 12 | N | 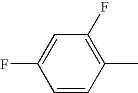 | CH₃ | H | BOC | S | 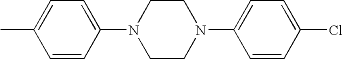 |
| 13 | N | 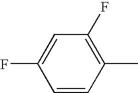 | CH₃ | H | BOC | S | 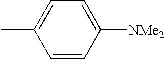 |
| 14 | N | 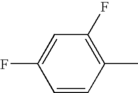 | CH₃ | H | BOC | S | 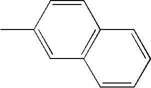 |
| 15 | N | 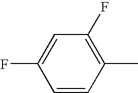 | CH₃ | H | BOC | S | 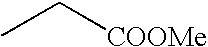 |
| 16 | N | 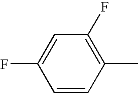 | CH₃ | H | BOC | S | 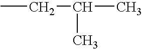 |
| 17 | N | 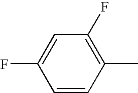 | CH₃ | H | BOC | S | 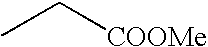 |
| 18 | N | 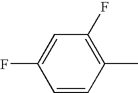 | CH₃ | H | BOC | S | 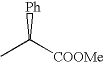 |
| 19 | N | 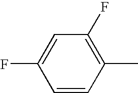 | CH₃ | H | BOC | S | 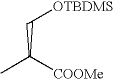 |
| 20 | N | 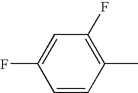 | CH₃ | H | BOC | S | 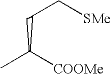 |
| 21 | N | 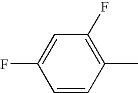 | CH₃ | H | BOC | S | 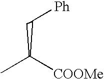 |
| 22 | N | 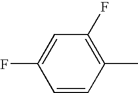 | CH₃ | H | BOC | S | 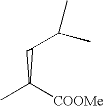 |
| 23 | N | 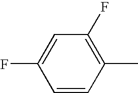 | CH₃ | H | BOC | S | 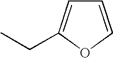 |
| 24 | N | 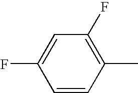 | CH₃ | H | BOC | S | 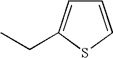 |
| 25 | N | 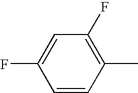 | CH₃ | H | BOC | O | 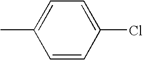 |
| 26 | N | 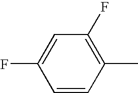 | CH₃ | H | BOC | O | 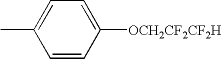 |
| 27 | N | 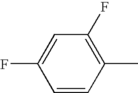 | CH₃ | H | BOC | O | 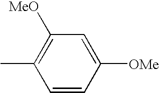 |
| 28 | N | 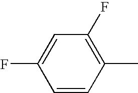 | CH₃ | H | H | O | 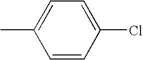 |
| 29 | N | 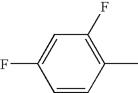 | CH₃ | H | H | S | 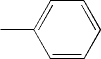 |
| 30 | N | 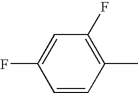 | CH₃ | H | H | S | 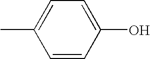 |
| 31 | N | 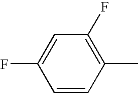 | CH₃ | H | H | S | 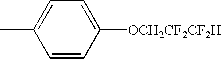 |
| 32 | N | 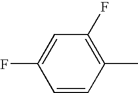 | CH₃ | H | H | S | 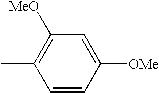 |
| 33 | N | 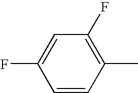 | CH₃ | H | H | S | 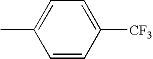 |
| 34 | N | 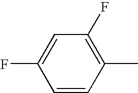 | CH₃ | H | H | S | 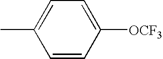 |
| 35 | N | 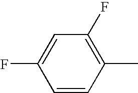 | CH₃ | H | H | S | 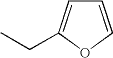 |
| 36 | N | 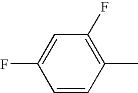 | CH₃ | H | H | S | 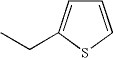 |
| 37 | N | 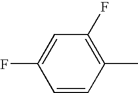 | CH₃ | H | H | S | 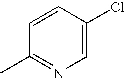 |
| 38 | N | 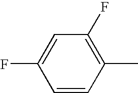 | CH₃ | H | H | S | 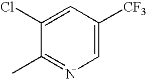 |
| 39 | N | 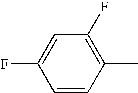 | CH₃ | H | H | S | 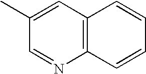 |

TABLE II

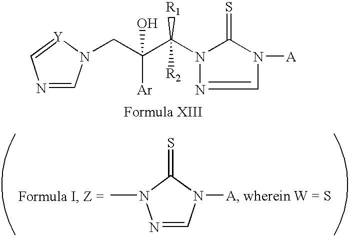

| Compound No. | Y | Ar | R₁ | R₂ | A |
|---|---|---|---|---|---|
| 40 | N | 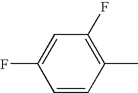 | CH₃ | H | 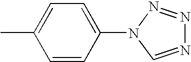 |
| 41 | N | 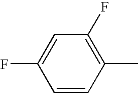 | CH₃ | H | 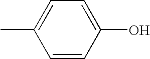 |
| 42 | N | 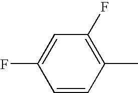 | CH₃ | H | 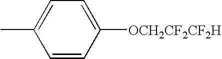 |
| 43 | N | 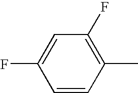 | CH₃ | H | 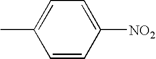 |
| 44 | N | 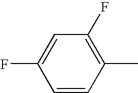 | CH₃ | H | 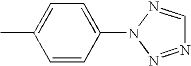 |
| 45 | N | 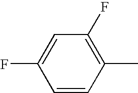 | CH₃ | H | 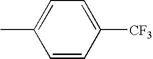 |
| 46 | N | 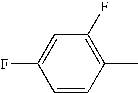 | CH₃ | H | 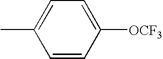 |
| 47 | N | 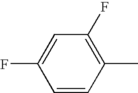 | CH₃ | H | 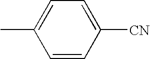 |
| 48 | N | 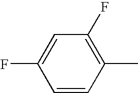 | CH₃ | H | CH₂COOMe |
| 49 | N | 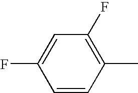 | CH₃ | H | 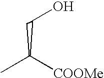 |
| 50 | N | 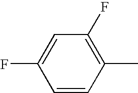 | CH₃ | H | 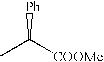 |
| 51 | N | 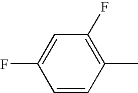 | CH₃ | H | 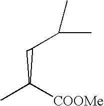 |
| 52 | N | 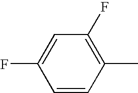 | CH₃ | H | 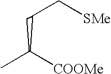 |
| 53 | N | 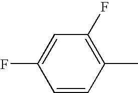 | CH₃ | H | 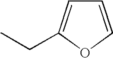 |
| 54 | N | 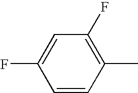 | CH₃ | H | 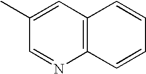 |
| 55 | N | 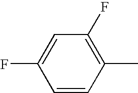 | CH₃ | H | 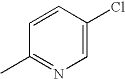 |
| 56 | N | 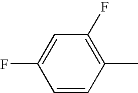 | CH₃ | H | 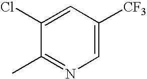 |

### DETAILED DESCRIPTION OF THE INVENTION

In order to achieve the above mentioned objectives and in accordance with the purpose of the invention as embodied and broadly described herein, there is provided a process for the synthesis of compound of Formula I, as show in Schemes I and II. The starting materials for Scheme I and Scheme II may be suitably adapted to produce the more specific compounds of Formula I. 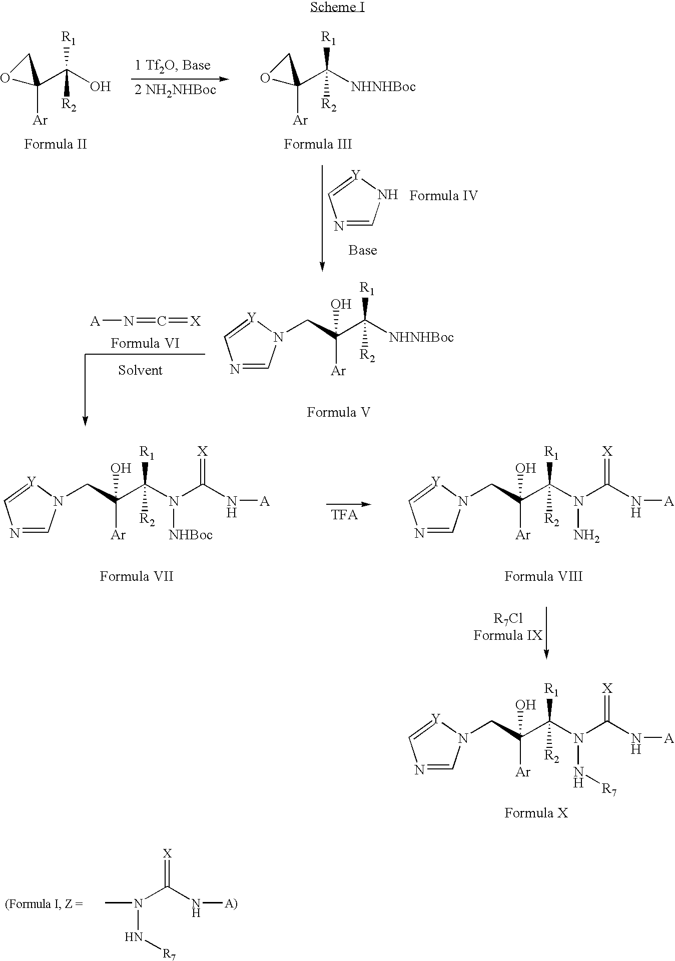
In Scheme I, there is provided a process for preparing a compound of Formula X (Formula I, when 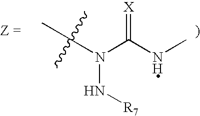
wherein
Ar is phenyl or a substituted phenyl having one to three substituents independently selected from halogen (chlorine, fluorine, bromine, iodine), nitro, cyano, lower (C₁₋₄)alkyl, lower(C₁₋₄)alkoxy, perhalo lower(C₁₋₄)alkyl or perhalo lower(C₁₋₄)alkoxy; five to seven membered heterocyclic ring containing one to four heteroatoms selected from the group consisting of oxygen, nitrogen and sulphur, the more preferred Ar is 2,4-difluorophenyl;
R₁ and R₂ are independently selected from the group consisting of hydrogen, straight chain or branched alkyl groups having 1 to 3 carbon atoms for example methyl, ethyl, propyl or isopropyl and their combinations thereof; the preferred alkyls are methyl and ethyl ; the more preferred combination is when R₁ is methyl and R₂ is hydrogen;
Y is CH or N;
X is selected from S, O, CH—NO₂, and N—CN;
A is hydrogen, unsubstituted or substituted lower (C₁₋₁₀)alkyl, said substituents being halogen (fluorine, chlorine, bromine or iodine), hydroxy, lower (C₁₋₄)alkoxy, lower (C₁₋₄)perhaloalkyl, lower (C₁₋₄)perhaloalkoxy; optionally substituted naphthyl; unsubstituted or substituted aromatic or non aromatic 5-6 membered rings with or without one to four heteroatoms independently selected from the group consisting of oxygen, nitrogen and sulphur, said substituents independently selected from one or more groups such as halogen (fluorine, chlorine, bromine or iodine), nitro, cyano, hydroxy, lower(C₁₋₄)alkyl, lower(C₁₋₄)alkoxy, lower (C₁₋₄)perhaloalkyl, lower (C₁₋₄)perhaloalkoxy, BR₃, substituted or unsubstituted five or six membered heterocyclic ring systems containing one to four heteroatoms selected from the group consisting of oxygen, nitrogen and sulphur, said heterocycylic substituents being (C₁-C₈)alkanoyl, lower (C₁-C₄)alkyl, lower (C₁-C₄)alkoxy carbonyl, N lower (C₁-C₄)alkylaminocarbonyl, N,N-dilower(C₁-C₄)alkylaminocarbonyl, N-lower (C₁-C₄)alkylaminothiocarbonyl, N,N-di(lower alkyl)(C₁-C₄)aminothiocarbonyl, N-lower (C₁-C₄)alkyl sulphonyl, phenyl substituted lower (C₁-C₄)alkyl sulphonyl, N-lower (C₁-C₄)alkyl amino, N,N-di(lower alkyl)(C₁-C₄)amino,unsubstituted or substituted phenyl, said substituents being halogen (fluorine, chlorine, bromine or iodine), hydroxy, lower (C₁₋₄)alkoxy, lower (C₁₋₄)perhaloalkyl, lower (C₁₋₄)perhaloalkoxy, nitro, cyano, amino, N(R₄)₂, 5-6 membered heterocyclic rings, the preferred heterocycles being 1,3-imidazolyl; 1,2,4 triazolyl ; —CHR₅R₆;
wherein
R₃ is a five or six membered aromatic or non aromatic ring with or without heteroatoms (oxygen, nitrogen and sulphur);
B is independently selected from (CH₂)ₘ, —O(CH₂)ₘ, —S(CH₂)ₘ;
m is an integer from 1 to 4;
R₄ is hydrogen, unsubstituted or substituted lower (C₁₋₄)alkyl;
R₅ is —COQ, where Q=OR₄, —N(R₄)₂;
R₆ is independently selected from hydrogen, straight chain or branched alkyl with or without substituents, said substituents being halogen (fluorine, chlorine, bromine or iodine), hydroxy, lower (C₁₋₄)alkyl, lower (C₁₋₄)alkoxy, lower (C₁₋₄0perhaloalkyl, lower (C₁₋₄)perhaloalkoxy, SR₄; phenyl or phenyl substituted with halogen (fluorine, chlorine, bromine or iodine), hydroxy, lower (C₁₋₄)alkoxy, lower (C₁₋₄)perhaloalkyl, lower (C₁₋₄)perhaloalkoxy, SR₄; heterocyclic rings or substituted heterocyclic rings with heteroatoms selected from oxygen, nitrogen and sulphur, substituents on heterocyclic rings are independently selected from halogen (fluorine, chlorine, bromine or iodine), hydroxy, lower (C₁₋₄)alkyl, lower (C₁₋₄)alkoxy, lower (C₁₋₄)perhaloalkyl, lower (C₁₋₄)perhaloalkoxy, SR₄; the preferred heterocyclic rings are imidazole and indole;
R₇ is H or selected from the group consisting of 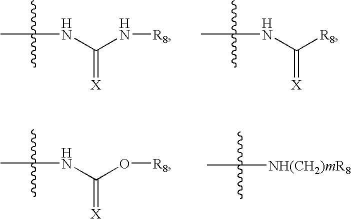
   wherein
R₈ is independently selected from hydrogen, unsubstituted or substituted lower (C₁₋₄)alkyl, aralkyl, aromatic or non aromatic 5-6 membered rings with or without one to four heteroatoms selected independently from the group consisting of oxygen, nitrogen and sulphur,
which comprises the conversion of the epoxy alcohol of Formula II to the corresponding triflate derivatives with trifluoromethane sulphonic anhydride (Tf₂O) in the presence of Hunig's base i.e. N,N-diisopropyl ethylamine, which is further subjected to nucleophilic substitution with t-butyl carbazate to afford substituted hydrazine of the Formula III with inversion of configuration at C-1, which on reaction with compound of Formula IV in the presence of a base gave epoxide ring opened intermediate of the Formula V which is then treated with a compound of the Formula VI to give Boc protected semicarbazide or thiosemicarbazide derivatives of the Formula VII which is further deprotected using trifluoroacetic acid to give the free amine of Formula VIII which may be treated with a compound of Formula IX to give a compound of Formula X (Formula I, when

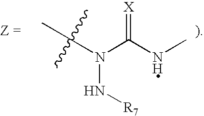

The starting compound of Formula II can be prepared by the process as described in U.S. Pat. No. 6,133,485.

The conversion of a compound of Formula II to the compound of Formula III is carried out in a solvent selected from the group consisting of chloroform, dichloromethane, dichloroethane, tetrahydrofuran, and the like. The reaction may be carried out in the presence of a base selected from the group consisting of triethylamine, Hunig's base, pyridine etc. The reaction temperature may range from −78° C. to 40° C. The nucleophilic epoxide ring opening of the compound of Formula IV may be carried out in the presence of a base such as potassium carbonate, cesium carbonate, calcium carbonate, sodium hydride, and the like. The reaction may be carried out in a solvent selected from the group consisting of dimethylformamide, dimethylsulfoxide, tetrahydrofuran, benzene, toluene, and the like or mixture(s) thereof. The reaction temperature may range from about 200 to 120° C., preferably at a temperature in the range of 80-85° C. The reaction of the compound of Formula V with a compound of Formula VI to give a compound of Formula VII, is carried out in an organic solvent that can be selected from the group consisting of chloroform, dichloromethane, dichloroethane and tetrahydrofuran at a temperature ranging from about 40-90° C. The deprotection of the Boc group in compound of Formula VII to give the free amine of Formula VII may be carried in an organic solvent such as chloroform, dichloromethane, dichloroethane, tetrahydrofuran, and the like at a temperature ranging from about 0-5° C. in the presence of trifluoroacetic acid. The reaction of compound of Formula VIII with a compound of Formula IX to give the compound of Formula X may be carried out in an organic solvent such as chloroform, dichloromethane, dichloroethane and tetrahydrofuran. The reaction temperature may range from about 0° C. to room temperature.

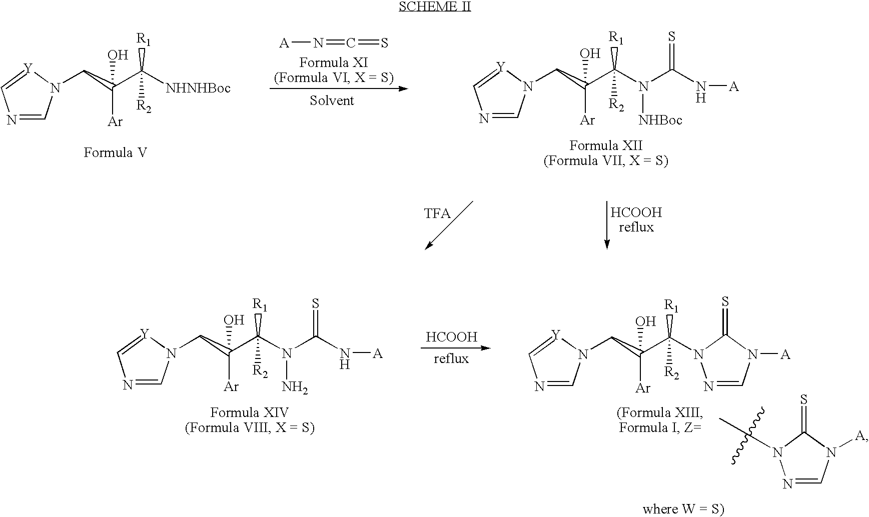

Scheme II shows the synthesis of compounds of Formula XIII (Formula I, when 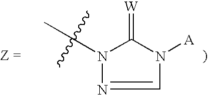
in which Ar, Y, R₁, R₂, W and A have the same meaning as defined earlier, which comprises treating the compound of Formula V with the isothiocyanate of Formula XI (Formula VI; X═S) and the resulting Boc derivatives of Formula XII (Formula VII; X═S) is further refluxed to give the desired compound of Formula XIII (Formula I, when 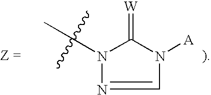
The free amine of Formula XIV

(Formula VIII, X═S) obtained by treating the compound of Formula XII with trifluoroacetic acid, upon refluxing, also gives the compound of Formula XIII.

The reaction of the compound of Formula V with the isothiocyanate of Formula XI may be carried out in an organic solvent such as chloroform, dichloromethane, dichloroethane tetrahydrofuran, and the like. The reaction temperature may range from about 40-90° C.

The deprotection of the BoC group in compound of Formula XII is carried out in the presence of an organic solvent selected from the group consisting of chloroform, dichloromethane, dichlorothane and tetrahydrofuran in the presence of trifluoroacetic acid.

The ring cyclization of the compound of Formula XII or its free amine of Formula XIV is carried out using formic acid at a temperature ranging from about 80-120° C.

In the above schemes, where specific bases, solvents, deprotecting agents etc. are mentioned, it is to be understood that other bases, solvents, deprotecting agents etc. known to those skilled in the art may also be used. Similarly, the reaction temperature and duration of the reactions may be adjusted according to the desired needs.

The intermediates of Formula III, V, VII and VIII are new and therefore they also constitute a further object of the invention. These intermediates are highly versatile and can be converted to a multitude of potential antifungal compounds.

The invention is explained in detail in the examples given below which are provided by way of illustration only and therefore should not be constrained to limit the scope of the invention.

### EXAMPLE 1

### Preparation of 1-t-butoxycarbonyl-2-[(1R,2R)-2-(2,4-difluorophenyl)-2-hydroxy-1-methyl-3-(1H-1,2,4-triazol-1-yl)propyl]4-[4-(2,2,3,3-tetrafluoropropoxy)phenyl]thiosemicarbazide (Compound No.7)

### Step a: Preparation of 2-[(1R,2R)-2-(2,4-difluorophenyl)-2,3-epoxy-1-methylpropyl]-1-t-butylcarbazate

In a dry 500 ml 3 neck round-bottom flask equipped with a nitrogen inlet, guard tube, addition funnel and a septum were placed the epoxy alcohol (10 g), Hunig's base (19 ml), and dichloromethane(60 ml). The mixture was cooled to −78° C. and trifluoromethanesulfonic anhydride (8.95 ml) was added dropwise. After the completion of addition, the reaction mixture was stirred at −78° C. for 30 minutes and at −20° C. for another 30 minutes. A solution of t-butyl carbazate (13 g) in tetrahydrofuran (30 ml) was then added to the above. The reaction mixture was further stirred at this temperature for 2 hours followed by stirring at room temperature for 18 hours. Tetrahydrofuran was evaporated and residue taken up in dichloromethane (150 ml). The organic layer was washed with water, brine and dried over sodium sulphate. The solvent was evaporated in vacuo and the residue was purified through column chromatography (silica gel, 100-200 mesh, 6:4 DCM:hexane) to afford the title compound (9.65 g, 61%).

¹H NMR (300 MHz, CDCl₃): δ 1.07 (d, J=6.7 Hz, 3H), 1.46 (s, 9H), 2.79 (d, J=5 Hz, 1H), 3.08 (d, J=5 Hz, 1H), 3.22 (q, J=6.7 Hz, 1H), 5.97 (s, 1H), 6.19 (s, 1H), 6.76-6.90 (m, 2H), 7.35-7.43 (m, 1H).

### Step b: Preparation of 2-[(1R,2R)-2-(2,4-difluorophenyl)-2-hydroxy-1-methyl-3-(1H-1,2,4-triazol-1-yl )propyl]-1-t-butylcarbazate

To a solution of epoxide (9.86 g) obtained in the previous step and 1,2,4-triazole (4.3 g) in dry N,N-dimethylformamide (50 ml) was added anhydrous K₂CO₃ (8.6) under nitrogen atmosphere. The reaction mixture was stirred at 40° C. for 15 hours and then at 70° C. for 4 hours. After the completion of reaction, the reaction mixture was poured in ice cold water (500 ml) and the organic layers were extracted into ethyl acetate (3×100 ml). The combined organic layers were washed with water, brine and dried over Na₂SO₄. The solvent was removed in vacuo and the residue obtained was purified through column chromatography (silica gel, 100-200 mesh, 20% EtOAc-DCM) to afford the title compound (7 g).

¹H NMR (300 MHz, CDCl₃): δ 0.91 (d, J=6.7 Hz, 3H), 1.48 (s, 9H), 3.52 (q, J=6.7 Hz, 1H), 4.75-4.90 (m, 3H), 6.2 (s, 1H), 6.70-6.77 (m, 2H), 7.33-7.41 (m, 1H), 7.73 (s, 1H), 7.90 (s, 1H).

### Step c: Preparation of 1-t-Butoxycarbonyl-2-[(1R,2R)-2-(2,4-difluorophenyl)-2-hydroxy-1-methyl-3-(1H-1,2,4-triazol-1-yl)propyl]4-[4-(2,2,3,3-tetrafluoropropoxy)phenyl]thiosemicarbazide (Compound No. 7)

To a solution of the amine (6.31 g) obtained in step (b) in 1,2-dichloroethane (30 ml) was added 4-[2,2,3,3-tetrafluoropropoxy]-phenylisothiocyanate and the mixture refluxed for 12 hours. After the completion of reaction, the solvent was evaporated and the residue obtained was purified through column chromatography (silicagel, 100-200 mesh, 10% EtOAc-DCM) to afford the title compound (7g, 78%).

¹H NMR (300 MHz, CDCl₃): δ 1.05 (d, J=6.6 Hz, 3H), 1.51 (s, 9H), 4.35 (t, J=11.7 Hz, 2H), 4.44 (d, J=14.5 Hz, 1H), 5.55 (d, J=14.3 Hz, 1H), 5.82 (s, 1H), 6.07 (tt, J=53.1 and 4.9 Hz, 1H), 6.74-6.79 (m, 3H), 6.93-6.96 (d, J=8.8 Hz, 2H), 7.36-7.39 (d, J=8.8 Hz, 2H), 7.79 (s, 1H), 7.81 (s, 1H), 8.51 (brs, 1H).

The illustrative list of the compounds of the invention which were synthesized by the above method is given below:

### 1-t-Butoxycarbonyl-2-[(1R,2R)-2-(2,4-difluorophenyl)-2-hydroxy-1-methyl-3-(1H -1,2,4-triazol-1-yl)propyl]-4-[4-fluorophenyl]thiosemicarbazide (Compound No.1)

¹H NMR (CDCl₃; 300 MHz):

δ: 1.06 (3H, d, J=6.1 Hz), 1.52 (9H, s), 4.44 (1H, d, J=15.4 Hz), 5.56 (1H, d, J=16.9 Hz), 5.83 (1H, s), 6.75-6.81 (3H, m), 7.06-7.11(2H, m), 7.33-7.43 (3H, m), 7.81 (2H, m), 8.52(1H, brs).

### 1-t-Butoxycarbonyl-2-[(1R,2R)-2-(2,4-difluorophenyl)-2-hydroxy-1-methyl-3-(1H -1,2,4-triazol-1-yl)propyl]-4-[2,4-difluorophenyl]thiosemicarbazide (Compound No. 2)

¹H NMR (CDCl₃; 300 MHz):

δ: 1.05 (3H, d, J=6.7 Hz), 1.51 (9H, s), 4.41 (1H, d, J=14.3 Hz), 5.55 (1H, d, J=13.5 Hz), 5.84 (1H, brs), 6.69-6.76 (3H, m), 6.86-6.94(2H, m), 7.29-7.37 (1H, brm), 7.79(1H, brs), 7.81(1H, brs), 7.9(1H, brs), 8.4 (1H,brs).

### 1-t-Butoxycarbonyl-2-[(1R,2R)-2-(2,4-difluorophenyl)-2-hydroxy-1-methyl-3-(1H -1,2,4-triazol-1-yl)propyl]-4-[4-trifluoromethylphenyl]thiosemicarbazide (Compound No. 3)

¹H NMR (CDCl₃; 300 MHz):

δ: 0.92 (3H, d, J=6.8 Hz), 1.51 (9H, s), 4.43 (1H, d, J=14.4 Hz), 5.53 (1H, d, J=13.9 Hz), 5.88 (1H, s), 6.73-6.8 (3H, m), 7.33-7.35 (1H, m), 7.62-7.7 (4H, m), 7.81(2H, s), 8.75 (1H, brs).

### 1-t-Butoxycarbonyl-2-[(1R,2R)-2-(2,4-difluorophenyl)-2-hydroxy-1-methyl-3-(1H -1,2,4-triazol-1-yl)propyl]-4-[2,4-dimethoxyphenyl]thiosemicarbazide (Compound No. 4)

¹H NMR (CDCl₃; 300 MHz):

δ: 1.04 (3H, d, J=6.9 Hz), 1.5 ( 3H, s), 3.8 (3H, s), 3.81 (3H,s), 4.43 (1H,d, J=14.5 Hz), 5.61 (1H, d, J=13.9 Hz), 5.72 (1H, s), 6.49-6.54 (2H, m), 6.71-6.80 (3H, m), 7.29-7.37 (1H, m), 7.78 (1H, s), 7.80(1H, s), 8.19 (1H, d, J=8.2 Hz), 8.8 (1H, s)

### 1-t-Butoxycarbonyl-2-[(1R,2R)-2-(2,4-difluorophenyl)-2-hydroxy-1-methyl-3-(1H -1,2,4-triazol-1-yl)propyl]-4-[4-(tetrahydropyranyloxy)phenyl]thiosemicarbazide (Compound No. 5)

¹H NMR (CDCl₃; 300 MHz):

δ: 1.06 (3H, d, J=5.9 Hz), 1.48 (9H, s), 3.6 (1H, d, J=11.2 Hz), 3.87-3.93 (1H, m), 4.34.8 (brm, 1H), 5.59 (1H,d, J=14.4 Hz), 6.72-6.8 (3H, m), 7.05-7.08 (2H, m), 7.31-7.37 (3H, m), 7.83 (2H, brs), 8.75 (1H, brs).

### 1-t-Butoxycarbonyl-2-[(1R,2R)-2-(2,4-difluorophenyl)-2-hydroxy-1-methyl-3-(1H-1,2,4-triazol-1-yl)propyl]-4-[4-trifluoromethoxyphenyl]thiosemicarbazide (Compound No. 6)

¹H NMR (CDCl₃): δ 1.08 (d, J=6.41 Hz, 3H, CHCH₃), 1.53 (bs, 9H, BOC—H), 4.44 (d, J=14.43 Hz, 1H, CH₂-Triazole), 5.56 (d, J=14.40 Hz, 1H, CH₂-Triazole), 5.88 (bs, 1H, D₂O-exchangeable, —OH), 6.75-6.83 (m, 3H, 2H of ArF₂ and 1H of CHCH₃), 7.24-7.28 (m, 2H ArOCF₃—H), 7.32-7.40 (m, 1H ArF₂—H), 7.55 (d, J=8.71 Hz 2H, 2H of ArOCF₃), 7.81 (s, 1H, Triazole-H), 7.83 (s, 1H, Triazole-H), 8.64 (bs, 1H, D₂O-exchangeable, —NH)

### 1-t-Butoxycarbonyl-2-[(1R,2R)-2-(2,4-difluorophenyl)-2-hydroxy-1-methyl-3-(1H -1,2,4-triazol-1-yl)propyl]-4-[4-nitrophenyl]thiosemicarbazide (Compound No. 8)

¹H NMR (CDCl₃; 300 MHz):

δ: 1.06 (3H, d, J=6.5 Hz), 1.51 (9H, s), 4.42 (1H, d, J=14.3 Hz), 5.50 (1H, d, J=13.6 Hz), 5.93 (1H, s), 6.74-6.82 (3H, m), 7.30-7.38 (1H, m), 7.80-7.83 (4H, m), 8.23(1H, s), 8.26 (1H, s), 8.92 (1H,s).

### 1-t-Butoxycarbonyl-2-[(1R,2R)-2-(2,4-difluorophenyl)-2-hydroxy-1-methyl-3-(1H -1,2,4-triazol-1-yl)propyl]-4-[4-(1,2,3,4-tetrazol-1-yl])phenyl]thiosemicarbazide (Compound No. 9)

¹H NMR (CDCl₃; 300 MHz):

δ: 1.07 (3H, d, J=6.5 Hz), 1.52 (9H, s), 4.45 (1H, d, J=14.3 Hz), 5.52 (1H, d, J=13.9 Hz), 5.91 (1H, s), 6.76-6.82 (3H, m), 7.31-7.39 (1H, m), 7.70-7.90 (6H, m), 8.80(1H, s), 9.0(1H, s).

### 1-t-Butoxycarbonyl-2-[(1R,2R)-2-(2,4-difluorophenyl)-2-hydroxy-1-methyl-3-(1H -1,2,4-triazol-1-yl)propyl]-4-[4-(1,2,3,4-tetrazol-2-yl)phenyl]thiosemicarbazide (Compound No.10)

¹H NMR (CDCl₃; 300 MHz):

δ: 1.07 (3H, d, J=6.5 Hz), 1.52 (9H, s), 4.46 (1H, d, J=14.4 Hz), 5.55 (1H, d, J=14.6 Hz), 5.89 (s, 1H), 6.74-6.82 (3H, m), 7.34-7.39 (1H, m), 7.75(2H, d, J=8.7 Hz), 7.80(1H, s), 7.82(1H, s), 8.17(1H, d, J=8.7 Hz), 8.66(1H, s), 8.77 (1H, brs).

### 1-t-Butoxycarbonyl-2-1(1R,2R)-2-(2,4-difluorophenyl)-2-hydroxy-1-methyl-3-(1H -1,2,4-triazol-1-yl)propyl]4-[4-cyanophenyl]thiosemicarbazide (Compound No.11)

¹H NMR (CDCl₃; 300 MHz):

δ: 1.05 (3H, d, J=6.5 Hz), 1.50 (9H, s), 4.41 (1H, d, J=14.3 Hz), 5.49 (1H, d, J=13.3 Hz), 5.92 (1H, s), 6.73-6.82 (3H, m), 7.30-7.38 (1H, m), 7.63-8.01 (7H, m), 8.85 (1H, brs).

### 1-t-Butoxycarbonyl-2-[(1R,2R)-2-(2,4-difluorophenyl)-2-hydroxy-1-methyl-3-(1H -1,2,4-triazol-1-yl)propyl]-4-[4-[4-chlorophenyl]-piperizinyl]phenyl]thiosemicarbazide (Compound No.12)

¹H NMR (CDCl₃; 300 MHz):

δ: 1.05 (3H, d, J=6.5 Hz), 1.51 (9H, s), 3.29-3.35(8H, m), 4.44 (1H, d, J=14.5 Hz), 5.56 (1H, d, J=13.6 Hz), 5.79 (1H,s), 6.73-6.79 (3H, m), 6.89(2H, d, J=9.0 Hz), 6.97(2H, d, J=8.9 Hz), 7.20-7.34 (5H, m), 7.79(1H, s), 7.81(1H, s), 8.49 (1H, s).

### 1-t-Butoxycarbonyl-2-[(1R,2R)-2-(2,4-difluorophenyl)-2-hydroxy-1-methyl-3-(1H-1,2,4-triazol-1-yl)propyl]-4[4-(N,N-dimethylamino)phenyl]thiosemicarbazide (Compound No.13)

¹H NMR (CDCl₃; 300 MHz):

δ: 1.05 (3H, d, J=6.3 Hz), 1.51(9H, s), 2.96(6H, s), 4.45(1H, d, J=14.4 Hz), 5.60 (1H, d, J=12.7 Hz), 5.71 (1H, s), 6.71-6.79 (5H, m), 7.22-7.25(2H, m), 7.30-7.38 (1H, m), 7.79(1H, s), 7.82(1H, s), 8.45(1H, brs).

### 1-t-Butoxycarbonyl-2-(1R,2R)-2-(2,4-difluorophenyl)-2-hydroxy-1-methyl-3-(1H -1,2,4-triazol-1-yl)propyl]-4-napthyl thiosemicarbazide (Compound No. 14)

¹H NMR (CDCl₃; 300 MHz):

δ: 1.1 (3H, m), 1.56 (9H, s), 4.56 (1H, s), 5.66 (1H, d, J=14.6 Hz), 5.87 (s, 1H), 6.73-6.79 (3H, m), 7.36-7.38 (1H, m), 7.52-7.62 (5H, m), 7.84-7.91(4H, m), 8.13(1H, brs), 8.75 (1H, brs).

### 1-t-Butoxycarbonyl-2-(1R,2R)-2-(2,4-difluorophenyl)-2-hydroxy-1-methyl-3-(1H-1,2,4-triazol-1-yl)propyl]-4-octyl thiosemicarbazide (Compound No.15)

¹H NMR (CDCl₃; 300 MHz):

δ: 0.85-0.89 (3H, m), 0.99(3H, d, J=6.6 Hz), 1.26 (10H, m), 1.51(9H, s), 3.55(1H, b s), 3.75-3.79(1H, m), 4.31 (1H, d, J=14.9 Hz), 5.57 (1H, d, J=14.2 Hz), 5.68 (s, 1H), 6.63-6.65 (1H, m), 6.70-6.79 (2H, m), 6.91(1H, brs), 7.29-7.35 (1H, m), 7.77(1H, s), 7.81 (1H, s).

### 1-t-Butoxycarbonyl-2-[(1R,2R)-2-(2,4-difluorophenyl)-2-hydroxy-1-methyl-3-1H 1,2,4-triazol-1-yl)propyl]-4-t-butyl thiosemicarbazide (Compound No. 16)

¹H NMR (CDCl₃; 300 MHz):

δ: 1.02 (3H, d, J=7.5 Hz), 1.49 (9H, s), 1.54 (9H, s), 4.31(1H, d, J=13.9 Hz), 5.46(1H, d, J=14.2 Hz), 5.64(1H, s), 6.73-6.79 (2H, m), 7.31-7.34 (1H, m), 7.83 (1H, s), 7.86(1H, s)

### Methyl-2-[1-t-butoxycarbonyl-2-[(1R,2R)-2-(2,4-difluorophenyl)-2-hydroxy-1-methyl-3-(1H -1,2,4-triazol-1-yl)propyl]thiosemicarbazid-4-yl]acetate (Compound No.17)

¹H NMR (CDCl₃; 300 MHz):

δ: 1.01 (3H, d, J=6.9 Hz), 1.5 (9H, s), 3.8 (3H, s), 4.36 (1H, d, J=14.4 Hz), 4.58 (2H, s), 5.39 (1H, d, J=14.6 Hz), 5.74 (1H, s), 6.56-6.71 (1H, m), 6.73-6.79 (2H, m), 7.3-7.32 (1H, s),7.35-7.4(1H, m), 7.77 (1H, s), 7.80(1H, s).

### Methyl-2-phenyl-2-[1-t-butoxycarbonyl-2-[(1R,2R)-2-(2,4-difluorophenyl)-2-hydroxy-1-methyl-3-(1H -1,2,4-triazol-1-yl)propyl]thiosemicarbazid-4-yl]acetate (Compound No. 18)

¹H NMR (CDCl₃; 300 MHz):

δ: 1.01 (3H, d, J=7.0 Hz), 1.44 (9H, s), 3.78 (3H, s), 4.43 (1H, d, J=14.4 Hz), 5.53 (1H, d, J=14.5 Hz), 5.77 (1H, s), 6.15 (1H, brs), 6.53-6.57 (1H, m), 6.72-6.77 (2H, m), 7.32-7.46 (4H, m), 7.70-7.73 (1H, m), 7.80 (1H, s), 7.83(1H, s).

### Methyl-2-[t-butyldimethylsilyloxymethyl]-2-[1-t-butoxycarbonyl-2-[(1R,2R)-2-(2,4-difluorophenyl)-2-hydroxy-1-methyl-3-(1H -1,2,4-triazol-1-yl)propyl]thiosemicarbazid-4-yl]acetate (Compound No.19)

¹H NMR (CDCl₃; 300 MHz):

δ:0.05 (6H, s), 0.88 (9H, s), 1.01(3H, d, J=6.5 Hz), 1.48 (9H, s), 3.72 (3H s,), 3.94-3.97 (1H, s), 4.18-4.21 (1H, m), 4.39-4.44 (1H, m), 5.26-5.30 (1H, m), 5.52-5.57 (1H, m), 5.75 (1H, s), 6.58-6.6 (1H, m), 6.73-6.78 (2H, m), 7.31-7.36 (2H, m), 7.71-7.74 (2H, m), 7.77(1H, s), 7.8 (1H, s)

### Methyl-2-[methylthioethyl]-2-[1-t-butoxycarbonyl-2-[(1R,2R)-2-(2,4-difluorophenyl)-2-hydroxy-1-methyl-3-(1H -1,2,4-triazol-1-yl)propyl]thiosemicarbazid-4-yl]acetate (Compound No. 20)

¹H NMR (CDCl₃; 300 MHz):

δ: 0.99 (3H, d, J=6.36 Hz), 1.49 (9H, s), 2.02-2.04 (2H, m), 2.10 (3H, s), 2.55-2.6 (2H, m) 3.76 (3H, s), 4.34 (1H, d, J=14.4 Hz), 5.26-5.32 (1H, m), 5.49 (1H, d, J=14.4 Hz), 5.74 (1H, s), 6.55-6.57(1H, m), 6.7-6.78 (2H, m), 7.3-7.32 (1H, m), 7.56(1H, brs), 7.71 (1H, brs), 7.77 (1H, s), 7.79 (1H, s).

### Methyl-2-benzyl-2-[1-t-butoxycarbonyl-2-[(1R,2R)-2-(2,4-difluorophenyl)-2-hydroxy-1-methyl-3-(1H -1,2,4-triazol-1-yl)propyl]thiosemicarbazid-4-yl]acetate (Compound No. 21)

¹H NMR (CDCl₃; 300 MHz):

δ: 1.03 (3H, d, J=6.0 Hz), 1.23-1.31 (3H, m), 1.52 (9H, s), 3.31-3.32 (2H, m), 4.12-4.19 (2H, m), 4.32 (1H, d, J=15 Hz), 5.46-5.57 (2H, m), 5.73 (1H, s), 6.61-6.63 (1H, m), 6.72-6.77 (2H, m), 7.20-7.22 (2H, m), 7.42-7.44 (1H, m), 7.65 (1H, brs), 7.77 (1H, s), 7.8(1H, s)

### Methyl-2-isobutyl-2-[1-t-butoxycarbonyl-2-[(1R,2R)-2-(2,4-difluorophenyl)-2-hydroxy-1-methyl-3-(1H -1,2,4-triazol-1-yl)propyl]thiosemicarbazid-4-yl]acetate (Compound No. 22)

¹H NMR (CDCl₃; 300 MHz):

δ: 0.94-1.01 (9H, m), 1.48 (9H, s), 1.64-1.77 (3H, m), 3.72 (3H, s), 4.34(1H, d, J=14.4 Hz), 5.11-5.16 (1H, m), 5.48 (1H, d, J=14.5 Hz), 5.73 (1H, s), 6.54-6.59 (1H, m), 6.70-6.77 (2H, m), 7.12 (1H, brs), 7.29-7.34 (1H, m), 7.76 (1H, s), 7.78 (1H, s).

### 1-t-Butoxycarbonyl-2-[(1R,2R)-2-(2,4-difluorophenyl)-2-hydroxy-1-methyl-3-(1H-1,2,4-triazol-1-yl)propyl]-4-[2-furanmethyl]thiosemicarbazide (Compound No. 23)

¹H NMR (CDCl₃): δ 1.00 (d, J=6.90 Hz, 3H, CHCH₃), 1.44 (bs, 9H, BOC—H), 4.32 (d, J=14.42 Hz, 1H, CH₂-Triazole), 4.89 (bs, 2H, CH₂-Furan), 5.53 (d, J=14.21 Hz, 1H, CH₂-Triazole), 5.70 (bs, 1H, D₂O-exchangeable, —OH), 6.33 (bs, 2H, Furan-H), 6.61-6.64 (m, 1H, CHCH₃), 6.70-6.79 (m, 2H, ArF₂—H), 7.29-7.35 (m, 1H,1H of furan), 7.76 (s,1H, Triazole-H), 7.80 (s,1H, Triazole-H)

### 1-t-Butoxycarbonyl-2-[1R,2R)-2-(2,4-difluorophenyl)-2-hydroxy-1-methyl-3-(1H-1,2,4-triazol-1-yl)propyl]-4-[2-thiophenmethyl]thiosemicarbazide (Compound No. 24)

¹H NMR (CDCl₃): δ 1.03 (d, J=6.85 Hz, 3H, CHCH₃), 1.46 (bs, 9H, BOC—H), 3.51 (bs, 1H, D₂O-exchangeable, —NH), 4.36 (d, J=14.45 Hz, 1H, CH₂-Triazole), 5.07 (bs, 2H, CH₂-Thiophene), 5.59 (d, J=14.59 Hz, 1H, CH₂-Triazole), 5.73 (bs, 1H, D₂O-exchangeable, —OH), 6.65-6.67 (m, 1H, CHCH₃), 6.73-6.82 (m, 2H, ArF₂—H), 6.97-7.00 (m, 1H, thiophene-H), 7.07 (bs, 2H, Thiophene-H), 7. 24-7.36 (m, 2H, 1H of thiophene and 1H of ArF₂), 7.63 (bs, 1H, D₂O-exchangeable, —NH), 7.79 (s, 1H, Triazole-H), 7.84 (s, 1H, Triazole-H)

### 1-t-Butoxycarbonyl-2-[(1R,2R)-2-(2,4-difluorophenyl)-2-hydroxy-1-methyl-3-(1H-1,2,4-triazol-1-yl)propyl]-4-[4-chlorophenyl]semicarbazide (Compound No. 25)

¹H NMR (CDCl₃; 300 MHz):

δ: 0.99 (3H, d, J=5.6 Hz), 1.51 (9H, s), 4.35 (1H, d, J=13.9 Hz), 5.2 (1H, d, J=13.7 Hz), 5.4(1H, brs), 5.59(1H, brs),6.73-6.79 (2H, m), 7.18-7.48 (5H, m), 7.54(1H, brs), 7.76(2H, s).

### 1-t-Butoxycarbonyl-2-[(1R,2R)-2-(2,4-difluorophenyl)-2-hydroxy-1-methyl-3-(1H-1,2,4-triazol-1-yl)propyl]-4-[4-(2,2,3,3-tetrafluoropropoxy)phenyl]semicarbazide (Compound No. 26)

¹H NMR (CDCl₃; 300 MHz):

δ: 1.51 (s, 9H), 4.32 (t, J=11.2 Hz, 3H) 5.21 (d, 11.4 Hz, 1H), 5.40 (brs, 1H), 5.57 (brs, 1H), 6.06 (tt, J=5.34 & 4.8 Hz, 1H), 6.73-6.79 (m, 2H), 6.88-6.91 (m, 2H), 7.26-7.43 (m,4H), 7.76 (s,1H)

### 1-t-Butoxycarbonyl-2-[(1R,2R)-2-(2,4-difluorophenyl)-2-hydroxy-1-methyl-3-(1H-1,2,4-triazol-1-yl)propyl]-4-[2,4-dimethoxyphenyl]semicarbazide. (Compound No.27)

¹H NMR (CDCl₃; 300 MHz):

δ: 1.00 (3H, d, J=6.3 Hz), 1.53 (9H, s), 3.79(3H, s), 4.4 (1H, d, J=14.5 Hz), 5.25(1H, d, J=13.4 Hz), 5.51-5.52 (1H, m), 6.47-6.49 (2H, m), 6.73-6.79(2H, m), 7.31-7.35(1H, m), 7.75(2H, m), 7.87(1H, s), 8.05(1H, d, J=7.0 Hz).

### 2-[(1R,2R)-2-(2,4-difluorophenyl)-2-hydroxy-1-methyl-3-(1H-1,2,4-triazol-1-yl)propyl]-4-[4-chlorophenyl]semicarbazide. (Compound No. 28)

¹H NMR (CDCl₃; 300 MHz):

δ: 1.06 (d, J=7.0 Hz, 3H), 4.63 (d, J=14.3 Hz, 1H), 5.24 (d, J=14.3 Hz, 1H), 5.30 (m, 1H), 6.72-6.83 (m, 2H), 7.24-7.34 (m, 2H), 7.45-7.47 (d, J=8.8 Hz, 2H), 7.84 (s,1H), 8.51 (brs, 1H), 8.91 (s, 1H).

### EXAMPLE 2

### Preparation of 2-[(1R,2R)-2-(2,4-difluorophenyl)-2-hydroxy-1-methyl-3-(1H-1,2,4-triazol-1-yl)propyl]-4-[4-(2,2,3,3-tetrafluoropropoxy)phenyl]thiosemicarbazide (Compound No. 31)

To a solution of compound no 1 (3.5 g in dichloromethane (30 ml) at 0° C. was slowly added a solution of trifluoroacetic acid in dichloromethane ( 30 ml, 30% v/v) and the reaction mixture was stirred at 0° C. temperature for 2 h. After the completion of reaction, the solvents were evaporated and the residue dissolved in dichloromethane. The organic layer was washed with 5% NaHCO₃ till no more effervescence was observed. The organic layer was washed with water, brine and dried over Na₂SO₄. The solvent was removed in vacuo and the residue purified through column chromatography (silica gel, 100-200 mesh, 10% EtOAc-DCM) to afford the title compound (1.81 g, 61%).

¹H NMR (300 MHz, CDCl₃): δ1.12 (d, J=7.0 Hz, 3H), 4.35 (t, J=11.8 Hz, 2H), 4.48 (d, J=14.6 Hz, 1H), 4.55 (s, 2H), 5.60 (d, J=14.6 Hz, 1H), 5.65 (s, 1H), 6.06 (tt, J=53.1 and 4.9 Hz, 1H), 6.64 (q, J=6.6 Hz, 1H), 6.73-6.80 (m, 2H), 6.94 (d, J=8.9 Hz, 2H), 7.33-7.36 (m, 1H), 7.46 (d, J=8.9 Hz, 2H), 7.79 (s, 1H), 7.83 (s,1H), 9.94 (s, 1H).

The illustrative list of the compounds of the invention which were synthesized by the above method is given below:

### 2-[(1R,2R)-2-(2,4-difluorophenyl)-2-hydroxy-1-methyl-3-(1H-1,2,4-triazol-1-yl)propyl]-4-phenyl thiosemicarbazide (Compound No. 29)

¹H NMR (CDCl₃; 300 MHz):

δ: 1.11 (3H, d, J=6.9 Hz), 3.49 (1H, s), 4.62 (1H, d, J=14.7 Hz), 5.60 (1H, d, J=14.5 Hz), 6.66-6.82 (3H, m), 7.18-7.23(1H, m), 7.31-7.39 (3H, m), 7.57-7.59(2H, m), 7.82 (1H, s), 8.36(1H, brs), 10.1(1H, brs).

### 2-[(1R,2R)-2-(2,4-difluorophenyl)-2-hydroxy-1-methyl-3-(1H-1,2,4-triazol-1-yl)propyl]-4-[4-hydroxyphenyl]thiosemicarbazide (Compound No. 30)

¹H NMR (CDCl₃; 300 MHz):

δ: 0.94 (3H, d, J=6.9 Hz), 4.51 (1H, d, J=14.5 Hz), 5.10 (1H, d, J=14.5 Hz), 6.51 (3H, q, J=6.9 Hz), 6.71(2H, d, J=8.7 Hz), 6.89-6.95(1H, m), 7.14-7.25(4H, m), 7.66 (1H, s), 8.31 (1H, s), 9.98(1H, brs).

### 2-[(1R,2R)-2-2,4-difluorophenyl)-2-hydroxy-1-methyl-3-(1H-1,2,4-triazol-1-yl)propyl]-4-12,4-dimethoxyphenyl]thiosemicarbazide (Compound No. 32)

¹H NMR (CDCl₃; 300 MHz):

δ: 1.11 (3H, d, J=6.9 Hz), 3.81 (3H, s), 3.83(3H, s), 4.49 (1H, d, J=14.7 Hz), 4.53(2H, s), 5.58 (1H, s), 5.64(1H, d, J=14.3 Hz), 6.51-6.54 (2H, m), 6.66-6.68(1H, m), 6.76-6.8(2H, m), 7.35-7.38 (1H, m), 7.78(1H, s), 7.84 (1H, s), 8.18(1H, d, J=8.5 Hz), 10.1(1H, s).

### 2-[(1R,2R)-2-(2,4-difluorophenyl)-2-hydroxy-1-methyl-3-(1H-1,2,4-triazol-1-yl)propyl]-4-[4-trifluoromethylphenyl ]thiosemicarbazide (Compound No. 33)

¹H NMR (CDCl₃; 300 MHz):

δ: 1.13 (3H, d, J=6.9 Hz), 4.46 (1H, d, J=14.4 Hz), 4.62(2H, s), 5.57 (1H, d, J=14.6 Hz), 5.7 (1H, s), 6.66-6.8 (1H, m), 6.77-6.82 (2H, m), 7.32-7.38 (1H, d, 7.61-7.63 (2H, m), 7.66-7.7 (2H, m), 7.8 (1H, s), 7.84 (1H, s), 10.34 (1H, s).

### 2-[(1R,2R)-2-(2,4-Difluorophenyl)-2-hydroxy-1-methyl-3-(1H-1,2,4-triazol-1-yl)propyl]-4-[4-trifluoromethoxyphenyl]thiosemicarbazide (Compound No. 34)

¹H NMR (CDCl₃): δ 1.14 (d, J=6.93 Hz, 3H, CHCH₃), 4.48 (d, J=15.00 Hz, 1H, CH₂-Triazole), 4.62 (bs, 2H, NH₂), 5.61 (d, J=15.00 Hz, 1H, CH₂-Triazole), 5.70 (bs, 1H, D₂O-exchangeable, —OH), 6.64-6.68 (m, 1H, CHCH₃), 6.77-6.84 (m, 2H, ArF₂—H), 7.20-7.29 (m, 2H, ArOCF₃-H), 7.34-7.42 (m, 1H, ArF₂—H), 7.66 (d, J=9.00 Hz, 2H, ArOCF₃—H), 7.82 (s, 1H, Triazole-H), 7.86 (s, 2H, Triazole-H), 10.17 (s, 1H, NH)

### 2-[(1R,2R)-2-(2,4-Difluorophenyl)-2-hydroxy-1-methyl-3-(1H-1,2,4-triazol-1-yl)propyl]-4-[2-furanmethyl]thiosemicarbazide (Compound No. 35)

¹H NMR (CDCl₃ ; 300 MHz) : δ: 1.05 (d, J=6.9 Hz, 3H, CH—CH₃), 4.33-4.41 (m, 3H, 1H of CH₂ Triazole and NH₂), 4.86 (d, J=5.1 Hz, 2H, CH₂ Furan), 5.54-5.59 (m, 2H, 1H of CH₂ Triazole and OH), 6.32-6.34 (m, 2H, Furan -H), 6.52 (q, 1H, J=6.6 Hz, CH—CH₃) 6.71-6.79 (m, 2H, ArF₂), 7.77 (s, 1H, Triazole —H), 7.84 (s, 1H, Triazole —H), 8.43 (brs, 1H, D₂O-exchangeable—NH).

### 2-[(1R,2R)-2-(2,4-Difluorophenyl) -2-hydroxy-1-methyl-3-(1H-1,2,4-triazol-1-yl)propyl]-4-[2-thiophenmethyl]thiosemicarbazide (Compound No. 36)

¹H NMR (CDCl₃; 300 MHz): δ: 1.05 (d, J=6.9 Hz, 3H, CH—CH₃), 4.32 (brs, 2H, D₂O-exchangeable, NH₂), 4.39 (d, J=14.7 Hz, 1H CH₂-Triazole), 5.04 (abq, J=15.3 Hz, 13.05 Hz, 2H, CH₂-Thiophene), 5.54 (brs, 1H, D₂O exchangaeable —OH), 5.59 (d, J=14.4 Hz, 1H, CH₂-Triazole), 6.50-6.54 (m, 1H, CH—CH₃), 6.72-6.80 (m, 2H, ArF₂—H), 6.95-6.98 (m, 1H, Thiophene-H), 7.05 (brs, 1H, Thiophene-H), 7.22-7.37 (m, 2H, 1H of Thiopene+1H of ArF₂—H), 7.76 (s, 1H, Triazole-H), 7.84 (s, 1H, Triazole-H), 8.45 (s, 1H, D₂O-exchangeable —NH)

### 2-[(1R,2R)-2-(2,4-Difluorophenyl)-2-hydroxy-1-methyl-3-(1H-1,2,4-triazol-1-yl)propyl]-4-[3-chloropyridin-6-yl]thiosemicarbazide (Compound No. 37)

¹H NMR (CDCl₃): δ 1.11 (d, J=6.90 Hz, 3H, CHCH₃), 4.43 (d, J=14.37 Hz, 1H, CH₂-Triazole), 4.64 (bs, 2H, —NH₂), 5.55 (d, J=14.37 Hz, 1H, CH₂-Triazole), 5.70 (bs, 1H, D₂O-exchangeable, —OH), 6.64-6.67 (m, 1H, CHCH₃), 6.74-6.81 (m, 2H, ArF₂—H), 7.31-7.36 (m, 1H, ArF₂—H), 7.66-7.70 (m, 1H, Pyridine-H), 7.77 (s, 1H, Triazole-H), 7.82 (s, 1H, Triazole-H), 8.27 (bs, 1H, Pyridine-H), 8.90 (d, J=8.91 Hz, 1H, Pyridine-H), 10.86 (bs, 1H, NH)

### 2-[(1R,2R)-2-(2,4-Difluorophenyl)-2-hydroxy-1-methyl-3-(1H-1,2,4-triazol-1-yl)propyl]-4-[5-chloro-3-trifluromethyl-pyridin-6-yl]thiosemicarbazide (Compound No. 38)

¹H NMR (CDCl₃): δ 1.14 (d, J=6.93 Hz, 3H, CHCH₃), 4.49 (d, J=14.46 Hz, 1H, CH₂-Triazole), 4.74 (bs, 2H, NH₂), 5.59 (d, J=14.58 Hz, 1H, CH₂-Triazole), 5.74 (bs, 1H, D₂O-exchangeable, —OH), 6.65-6.67 (m, 1H, CHCH₃), 6.74-6.81 (m, 2H, ArF₂—H), 7.30-7.40 (m, 1H, ArF₂—H), 7.79 (s, 1H, Triazole-H), 7.83 (s, 1H, Triazole-H), 7.99 (s, 1H, pyridine-H), 8.72 (s, 1H, pyridine-H), 10.72 (bs, 1H, NH),

### 2-[(1R,2R)-2-(2,4-Difluorophenyl)-2-hydroxy-1-methyl-3-(1H-1,2,4-triazol-1-yl)propyl]-4-[quinolin-3-yl]thiosemicarbazide (Compound No. 39)

¹H NMR (DMSO-d₆): δ 1.01 (d, J=6.67 Hz, 3H, CHCH₃), 4.59 (d, J=14.37 Hz, 1H, CH₂-Triazole), 5.15 (d, J=14.58 Hz, 1H, CH₂-Triazole), 6.33 (bs, 1H, D₂O-exchangeable —OH), 6.52-6.54 (m, 1H, CHCH₃), 6.92-6.97 (m,1H, ArF2H), 7.30-7.17 (m, 2H, ArF₂—H), 7.58-7.63 (m, 1H, quinoline-H), 7.68-7.74 (m, 2H, quinoline-H), 7.96-8.01 (m, 2H, 1H of Triazole and 1H of quinoline), 8.31 (s, 1H, triazole —H), 8.60 (s, 1H, quinoline-H), 9.00 (s, 1H, quinoline-H)

### EXAMPLE 3

### 2-[[1R,2R]-2-(2,4-difluorophenyl)-2-hydroxy-1-methyl-3-(1,2,4-triazol-1-yl)propyl]-4-[4-(2,2,3,3-tetrafluoropropoxy)phenyl]-3-(2H,4H)-1,2,4-triazol-3-thione (Compound No.42)

Method I: A solution of Compound No.24 (280 mg) in formic acid (0.6 ml) was -refluxed for 2 hours. After the completion of reaction, the reaction mixture was poured in ice cold water and neutralized with NaHCO₃. The organic layers were extracted into EtOAc, washed with water and dried over NaSO₄. Solvent was s removed in vacuo and the residue purified through column chromatography (silica gel, 100-200 mesh, 50% EtOAc-DCM) to afforded title compound (200 mg, 70%).

Method II: A solution of Compound No.1 (300 mg) in formic acid (2 ml) was refluxed for 1.5 hours. After completion of reaction, the reaction mixture was poured into ice cold water and neutralised with NaHCO₃. The organic layers were extracted with ethyl acetate and washed with water and dried over Na₂SO4. Solvent was removed in vacuo and the residue purified through column chromatography (silica gel, 100-200 mesh, 50% EtOAc-DCM) to afforded title compound,121 mg (50%)

¹H NMR (300 MHz, CDCl₃): δ1.33(d, J=6.9 Hz, 3H), 4.34-4.46 (m, 3H), 5.13 (d, J=14.4 Hz, 1H), 5.21 (s, 1H), 5.88-5.96 (m, 1H), 6.06 (tt, J=48.5 and 4.6 Hz, 1H), 6.82-6.88 (m, 2H), 7.09-7.12 (m, 2H), 7.53-7.65 (m, 3H), 7.74 (s, 1H), 7.93 (s, 1H).

The illustrative list of the compounds of the invention which were synthesized by the above method is given below:

### 2-[(1R,2R)-2-(2,4-difluorophenyl)-2-hydroxy-1-methyl-3-(1H-1,2,4-triazol-1-yl)propyl]-4-[4-(1,2,3,4-tetrazol-1-yl)phenyl]-(2H,4H)-1,2,4-trizol-3-thione (Compound No. 40)

¹H NMR (CDCl₃; 300 MHz): δ:1.35 (3H, d, J=6.9 Hz), 4.43 (1H, d, J=14.3 Hz), 5.07 (1H, d, J=14.3 Hz), 5.23 (1H, s), 5.94 (1H, q, J=7.08 Hz), 6.82-6.89 (2H, m), 7.59-7.67 (1H, m), 7.76 (1H, s), 7.89-8.02 (4H, m), 8.25 (1H, s), 9.0 (1H, s)

### 2-[(1R,2R)-2-(2,4-difluorophenyl)-2-hydroxy-1-methyl-3-(1H-1,2,4-triazol-1-yl)propyl]-4-[4-hydroxyphenyl]-(2H,4M)-1,2,4-triazol-3-thione (Compound No. 41)

¹H NMR (CDCl₃; 300 MHz): δ: 1.19 (3H, d, J=6.9 Hz), 4.28 (1H, d, J=14.4 Hz), 5.09 (2H, d, J=14.5 Hz), 5.74-5.80 (2H, m), 6.90-6.95 (3H, m), 7.18-7.35 (2H, m), 7.43 (2H, d, J=8.64 Hz), 7.59(1H, s), 8.30 (1H, s), 8.88 (1H,s), 9.97 (1H, s).

### 2-[(1R,2R)-2-(2,4-difluorophenyl)-2-hydroxy-1-methyl-3-(1H-1,2,4-triazol-1-yl)propyl]-4-[4-nitrophenyl]-(2H,4H)-1,2,4-triazol-3-thione (Compound No. 43)

¹H NMR (CDCl₃; 300 MHz): δ: 1.27 (3H, d, J=7.2 Hz), 4.42 (1H, d, J=14.3 Hz), 5.11 (1H, d, J=14.3 Hz), 5.21 (1H, s), 5.89-5.95 (1H, s), 6.82-6.88 (2H, m), 7.58-7.64 (1H, m), 7.75 (1H, s), 7.89 (1H, s), 7.91-7.93 (2H, m), 8.01 (1H, s), 8.44 (1H d, J=8.6 Hz).

### 2-[(1R,2R)-2-)2,4-difluorophenyl)-2-hydroxy-1-methyl-3-(1H-1,2,4-triazol-1-yl)propyl]-4[4-(1,2,3,4-tetrazol-2-yl)phenyl]-(2H,4H-1,2,4-triazol-3-thione (Compound No. 44)

¹H NMR (CDCl₃; 300 MHz): δ: 1.36 (3H, d, J=6.9 Hz), 4.42 (1H, d, J=14.4 Hz), 5.14 (1H, d, J=14.1 Hz), 5.22 (1H, s), 5.94-5.99 (1H, m), 6.79-6.89 (2H, m), 7.53-7.68 (1H, m), 7.74 (1H, s),7.77-7.86 (2H, m), 7.89 (1H, s), 7.95 (1H, s), 8.40 (2H, d, J=8.9 Hz), 8.72 (1H, s).

### 2-[(1R,2R)-2-(2,4-difluorophenyl)-2-hydroxy-1-methyl-3-(1H-1,2,4-triazol-1-yl)propyl]-4-[4-trifluoromethylphenyl]-(2H,4H)-1,2,4-triazol-3-thione (Compound No. 45)

¹H NMR (CDCl₃; 300 MHz): δ:1.35 (3H, d, J=6.9 Hz), 4.4 (1H, d, J=14.5 Hz), 5.13 (1H, d, J=14.2 Hz), 5.21 (1H, s), 5.93-5.95 (1H, m), 6.82-6.88 (2H, m), 7.58-7.63 (1H, s), 7.75(1H, s), 7.77-7.87 (5H, m), 7.93 (1H, s), 7.98 (1H, s).

### 2-[(1R,2R)-2-(2,4-Difluorophenyl)-2-hydroxy-1-methyl-3-(1H-1,2,4-triazol-1yl)propyl]-4-[4-trifluoromethoxyphenyl]-(2H,4H)-1,2,4-triazol-3-thione (Compound No. 46)

¹H NMR (CDCl₃): δ 7.96 (s, 2H, Triazole-H), 7.75 (s, 1H, Thio-triazolone-H), 7.68-7.57 (m, 3H, 1H of ArF₂ and 2H of ArOCF₃), 7.41 (d, 2H, J=8.46 Hz, 2H of ArOCF₃), 6.88-6.82 (m, 2H, 2H of ArF₂), 5.93 (q, 1H, J=6.96 Hz, CHCH₃), 5.20(bs, 1H, D₂O-exchangeable, —OH), 5.13 (d, 1H, J=14.37 Hz, CH₂-Triazole), 4.38 (d,1H, J=14.28 Hz, CH₂-Triazole), 1.33 (d, 3H, J=6.93 Hz, CHCH₃)

### 2-[(1R,2R)-2-(2,4-difluorophenyl)-2-hydroxy-1-methyl-3-(1,2,4-triazol-1-yl)propyl]-4-[4-cyanophenyl]( 2H,4H)-1,2,4-triazol-3-thione (Compound No. 47)

¹H NMR (CDCl₃; 300 MHz):

δ: 1.34 (3H, d, J=6.8 Hz), 4.41 (1H, d, J=14.3 Hz), 5.12(1H, d, J=14.3 Hz), 5.22-5.29(1H,m), 5.93 (1H, q, J=6.9 Hz),6.83-6.89(2H,m), 7.58-7.67(1H, m), 7.76(1H,s), 7.82-7.90(4H, m), 7.93(1H, s), 7.99(1H, s).

### Methyl-2-[[(1R,2R)-2-(2,4-difluorophenyl)-2-hydroxy-1-methyl-3-(1,2,4-triazol-1-yl)propyl]-(2H,4H)-1,2,4-triazol-3-thion-4-yl]acetate (Compound No. 48)

¹H NMR (CDCl₃; 300 MHz): δ:1.25 (3H, d, J=6.9 Hz), 3.79 (3H, s), 4.04 (1H, d, J=14.3 Hz), 4.87 (2H, q, 17.7 Hz), 4.98 (1H, d, 14.3 Hz), 5.81-5.83 (1H, m), 6.78-6.84 (2H, m), 7.50-7.56 (1H, m), 7.71 (1H, s), 7.89(1H, s), 8.03 (1H, s).

### Methyl-2-hydroxymethyl-2-[[(1R,2R)-2-(2,4-difluorophenyl)-2-hydroxy-1-methyl-3-(1H-1,2,4-triaolyl)propyl]-(2H, 4H)-1,2,4-triazol-3-thion-4-yl]acetate (Compound No. 49) [1:1 Mixture of diasteromers at C-2]

¹H NMR (CDCl₃; 300 MHz): δ 0.95 (3H, d, J=6.6 Hz), 1.31 (3H, d, J=7.0 Hz), 3.56-3.59 (2H, m), 3.74-3.82 (2H, m), 3.84 (3H, s), 3.88 (3H, s), 4.06 (1H, d, J=14.3 Hz), 4.4-4.49 (1H, m), 4.5-4.6 (1H, m), 5.0-5.18 (3H, m), 5.3-5.45 (1H, m), 5.82 (1H, q, J=7.0 Hz), 6.05 (1H, brs), 6.78-6.84 (4H, m), 6.98 (1H, s), 7.49-7.6 (1H, m), 7.69 (1H, s), 7.71 (1H, s), 7.83 (1H, s), 7.87 (1H, s), 8.08 (1H, s), 8.25 (1H, s)

### Methyl-2-phenyl-2-[[(1R,2R)-2-(2,4-difluorophenyl)-2-hydroxy-1-methyl-3-(1H-1,2,4-triaolyl)propyl]-(2H,4H-1,2,4-triazol-3-thion-4-yl]acetate (Compound No. 50) [1:1 Mixture of diasteromers at C-2]

¹H NMR (CDCl₃; 300 MHz): δ: 0.90 (3H, d, J=6.7 Hz), 1.22 (3H, d, J=7.4 Hz), 3.77 (3H, s), 3.86 (3H, s), 4.05 (1H, d, J=14.4 Hz), 4.53 (1H, d, J=15.1 Hz), 4.86 (1H, d, J=15.1 Hz), 5.18 (1H, d, J=14.5 Hz), 5.84 (1H, q, J=6.9 Hz), 6.35-6.45 (1H, m), 6.59 (1H, s), 6.79-6.82 (2H, m), 6.90-7.0 (2H, m), 7.05 (1H, s), 7.1-7.25 (1H, m), 7.38-7.50 (10H, m), 7.70-7.74 (3H, m), 7.82 (1H, s), 7.88 (1H, s)

### Methyl-2-isobutyl-2-[[(1R,2R)-2-2,4-difluorophenyl)-2-hydroxy-1-methyl-3-(1H-1,2,4-triaolyl)propyl]-(2H,4H)-1,2,4-triazol-3-thion-4-yl]acetate (Compound No. 51) [1:1 Mixture of diasteromers at C-2]

¹H NMR (CDCl₃; 300 MHz): δ: 0.92-1.04 (12H, m), 1.25-1.31 (6H, m), 1.53-1.59 (2H, m), 1.73-1.78 (2H, m), 1.79-2.00 (1H, m), 2.04-2.09 (1H, m), 3.78 (3H, s), 3.82 (3H, s), 4.01 (1H, d, J=14.4 Hz), 4.47 (1H, d, J=15.0 Hz), 4.83(1H, d, J=15.2 Hz), 5.11-5.19 (2H, m), 5.60-5.75 (1H, m), 5.85-5.95 (1H, m), 6.20-6.49 (1H, m), 6.81-6.85 (3H, m), 6.9-7.0 (1H, m), 7.05 (1H, s), 7.19-7.21 (1H, m), 7.45-7.55 (1H, m), 7.69 (1H, s), 7.70 (1H, s), 7.73 (1H, s), 7.87 (1H, s), 8.08 (1H, s)

### Methyl-2-methylthioethyl-2-[[(1R,2R)-2-(2,4-difluorophenyl)-2-hydroxy-1-methyl-3-(1H-1,2,4-triaolyl)propyl]-(2H,4H)-1,2,4-triazol-3-thion-4-yl]acetate (Compound No. 52) [1:1 Mixture of diasteromers at C-2]

¹H NMR (CDCl₃; 300 MHz): δ: 0.93 (3H, d, J=6.6 Hz), 1.31 (3H, d, J=6.9 Hz), 2.12 (6H, s), 3.81 (3H, s), 3.84 (3H, s), 4.04 (1H, d, J=14.4 Hz), 4.47 (1H, d, J=15.1 Hz), 4.83 (1H, d, J=15.1 Hz), 5.06 (1H, brs), 5.16 (1H, d, J=14.3 Hz), 5.25-5.30 (1H, m), 5.74-5.77 (1H, m), 5.84 (1H, q, J=7.0 Hz), 6.36 (1H, q, J=6.8 Hz), 6.78-6.85 (2H, m), 6.92-6.93 (2H, m), 7.07 (1H, s), 7.50-7.56 (1H, m), 7.70 (1H, s), 7.73 (1H, s), 7.87 (1H, s), 8.13 (1H, s).

### 2-[(1R,2R)-242,4-Difluorophenyl)-2-hydroxy-1-methyl-3-(1H-1,2,4-triazol-1-yl)propyl]-4-[2-furanmethyl]-(2H,4H)-1,2,4-triazol-3-thione (Compound No. 53).

¹H NMR (CDCl₃): δ 7.78 (s, 1H, Triazole-H), 7.83 (s, 1H, Triazole-H), 7.67 (s, 1H, Thio-triazolone-H), 7.56-7.47 (m, 2H, 1H of furan and 1H of ArF₂), 6.85-6.77 (m, 2H, ArF₂—H), 6.59 (bs, 1H, Furan-H), 6.42 (bs, 1H, Furan-H), 5.83(q, 1H, J=6.91 Hz, CHCH₃), 5.22 (s, 2H, Furan-H), 5.12 (d, 1H, J=14.48 Hz, CH₂-Triazole), 5.07 (bs, 1H, D₂O-exchangeable, —OH), 4.11 (d, 1H, J=14.20 Hz, CH₂-Triazole), 1.26 (d, 3H, J=6.94 Hz, CHCH₃)

MS (+ve ion): m/z 523.2 (M⁺+1)

### 2-[(1R,2R)-2-(2,4-Difluorophenyl)-2-hydroxy-1-methyl-3-(1H-1,2,4-triazol-1-yl)Propyl]-4-[quinolin-3-yl]-(2H,4H)-1,2,4-triazol-3-thione (Compound No. 54)

¹H NMR (DMSO-d₆): δ 9.18 (s, 1H, Quinoline-H), 9.15 (s, 1H, Quinoline-H), 8.77 (s, 1H, triazole —H), 8.32 (s, 1H, Triazole-H), 8.17-8.12 (m, 2H, Quinoline-H), 7.94-7.89 (m, 2H, Quinoline-H) 7.61 (s, 1H, Thio-triazolone-1H), 7.38-7.30 (m, 1H, ArF₂—H), 7.27-7.20 (m, 1H, ArF₂—H), 6.97-6.92 (m, 1H, ArF₂—H), 5.89 (bs, 1H, D₂O-exchangeable, —OH), 5.85-5.80 (m, 1H, CHCH₃), 5.11 (d, 1H, J=14.49 Hz, CH₂-Triazole), 4.39 (d, 1H, J=14.40 Hz, CH₂-Triazole), 1.25 (d, 3H, J=6.87 Hz, CHCH₃)

MS (+ve ion): m/z 479.9 (M⁺+1) 2-[(1R,2R)-2-(2,4-Difluorophenyl)-2-hydroxy-1-methyl-3-(1H-1,2,4-triazol-1-yl)propyl]-4-[3-chloropyridin-6-yl]-(2H,4H)-1,2,4-triazol-3-thione (Compound No. 55).

¹H NMR (CDCl₃): δ 9.02 (d, 1H, J=8.79 Hz, pyridine-H), 8.72 (s, 1H, pyridine-H), 8.48 (s, 1H, Thio-triazolone-H), 7.93-7.90 (m, 2H, 1H of triazole and 1H of pyridine), 7.70(s, 1H, triazole), 7.60-7.54 (m, 1H, ArF₂—H), 6.87-6.80 (m, 2H, ArF₂—H), 5.98 (q, 1H, J=6.96 Hz, CHCH₃), 5.17 (d, 1H, J=14.40 Hz, CH₂-Triazole), 5.10(bs, 1H, D₂O-exchangeable, —OH), 4.25 (d, 1H, J=14.28 Hz, CH₂-Triazole), 1.32 (d, 3H, J=6.93 Hz, CHCH₃)

MS (+ve ion): m/z 464.2 (M⁺+1)

### 2-[(1R,2R)-2-(2,4-Difluorophenyl)-2-hydroxy-1-methyl-3-(1H-1,2,4-triazol-1-yl)propyl]-4-[5-chloro-3-trifluoromethylpyridin-6-yl]-(2H,4H)-1,2,4-triazol-3-thione (Compound No. 56)

¹H NMR (CDCl₃): δ 7.78 (s, 1H, Triazole-H), 7.83 (s, 1H, Triazole-H), 7.67 (s, 1H, Thio-triazolone-H), 7.56-7.47 (m, 2H, 1H of furan and 1H of ArF₂), 6.856.77 (m, 2H, ArF₂—H), 6.59 (bs, 1H, Furan-H), 6.42 (bs, 1H, Furan-H), 5.83(q, 1H, J=6.91 Hz, CHCH₃), 5.22 (s, 2H, Furan-H), 5.12 (d, 1H, J=14.48 Hz, CH₂-Triazole), 5.07 (bs, 1H, D₂O-exchangeable, —OH), 4.11 (d, 1H, J=14.20 Hz, CH₂-Triazole), 1.26 (d, 3H, J=6.94 Hz, CHCH₃)

MS (+ve ion): m/z 523.2 (M⁺+1)

Antifungal Activity

The compounds of the Formula I and its salts are useful in the curative or prophylactic treatment of fungal infections in animals, including human.

The in vitro evaluation of the antifungal activity of the compound of this invention (as shown in Table I) can be performed by determining the minimum inhibitory concentration (MIC) which is the concentration of the test compound in Rosewell Park Memorial Institute (RPMI) 1640 liquid medium buffered with 3-(Morpholino)propane sulfonic acid (MOPS) to pH 7, at which there is significant inhibition of the particular fungi. In practice the National Committee for Clinical Laboratory Standard (NCCLS) M27A document for Candida and Cryptococcus and M38P for Aspergillus was used to determine the MIC and readings recorded only when the Quality Control results fell into the acceptable range. After MIC results had been recorded, 100 μL from each of the well showing no growth was spread over Sabouraud Dextrose Agar (SDA) to determine the minimum fungicidal concentration (MFC).

The results of in vitro tests are listed in Table III.

TABLE III
In vitro screening results of the synthesized compounds

| | |
|---|---|
| | MIC (A. fum.) |
| Compound No. | (μg/ml) |

| | | |
|---|---|---|
| | 1008 | Si-l |
| 1 | >16 | >16 |
| 2 | >16 | >16 |

| | |
|---|---|
| 3 | PS-VE |

| | | |
|---|---|---|
| 4 | >16 | >16 |
| 5 | >16 | >16 |
| 6 | 8 | 8 |
| 7 | 4 | 4 |
| 8 | >16 | >16 |
| 9 | >16 | >16 |
| 10 | >16 | >16 |

| | |
|---|---|
| 11 | PS-VE |

| | | |
|---|---|---|
| 12 | 0.25 | 0.25 |

| | |
|---|---|
| 13 | PS-VE |

| | | |
|---|---|---|
| 14 | >16 | >16 |
| 15 | >16 | >16 |

| | |
|---|---|
| 16 | PS-VE |
| 17 | PS-VE |
| 18 | PS-VE |
| 19 | PS-VE |
| 20 | PS-VE |
| 21 | PS-VE |
| 22 | PS-VE |
| 23 | PS-VE |
| 24 | PS-VE |

| | | |
|---|---|---|
| 25 | >16 | >16 |
| 26 | >16 | >16 |
| 27 | >16 | >16 |
| 28 | >16 | >16 |
| 29 | >16 | >16 |
| 30 | >16 | >16 |
| 31 | 1 | 0.25 |
| 32 | >16 | >16 |
| 33 | 4 | 2 |
| 34 | 2 | 2 |
| 35 | 4 | 4 |
| 36 | 4 | 2 |
| 37 | 0.5 | 1 |

| | |
|---|---|
| 38 | PS-VE |

| | | |
|---|---|---|
| 39 | 16 | 4 |

| | |
|---|---|
| 40 | PS-VE |

| | | |
|---|---|---|
| 41 | >16 | >16 |
| 42 | 0.25 | 0.125 |
| 43 | 4 | 2 |
| 44 | 2 | 2 |
| 45 | 1 | 0.5 |
| 46 | 2 | 0.5 |

| | |
|---|---|
| 47 | PS-VE |
| 48 | PS-VE |
| 49 | PS-VE |

| | | |
|---|---|---|
| 50 | >16 | >16 |
| 51 | >16 | <16 |
| 52 | >16 | >16 |
| 53 | 8 | 4 |

| | |
|---|---|
| 54 | PS-VE |

| | | |
|---|---|---|
| 55 | 4 | 2 |

| | |
|---|---|
| 56 | PS-VE |

The in vivo evaluation of the compound can be carried out at a series of dose levels by oral or I.V. injection to mice which are inoculated I.V. with the minimum lethal dose of Candida albicans, Cryptococcus neoformans or Aspergillus fumigatus by the tail vein. Activity is based on the survival of a treated group of mice after the death of an untreated group of mice. For Aspergillus and Cryptococcus infections, target organs were cultured after treatment to document the number of mice cured of the infection for further assessment of activity.

For human use, the antifungal compound of the present invention and its salts can be administered as above, but will generally by administered in admixture with a pharmaceutical carrier selected with regard to the intended route of administration and standard pharmaceutical practice for example, they can be administered orally in the form of tablets containing such excipients as starch or lactose or in capsules or ovules either alone or in admixture with exciepients or in the form of elixirs, solutions or suspensions containing flavouring or colouring agents. They can be injected parenterally, for example, intravenously, intramuscularly or sub-cutaneously. For parenteral administration they are best used in the form of a sterile aqueous solution which may contain other substances, for example, enough salts or glucose to make the solution isotonic with blood.

While the present invention has been described in terms of its specific embodiments, certain modifications and equivalents will be apparent to those skilled in the art and are intended to be included within the scope of the present invention.

We Claim:

1. A compound having the structure of Formula I 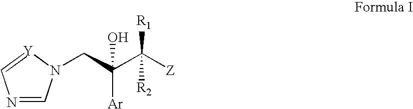
and its pharmaceutically acceptable salts, esters, enantiomers, diastereomers, N-oxides, prodrugs, metabolites, polymorphs and pharmaceutically acceptable solvates,
wherein
Ar is phenyl or a substituted phenyl having one to three substituents independently selected from halogen (chlorine, fluorine, bromine, iodine), nitro, cyano, lower(C₁₋₄)alkyl, lower(C₁₋₄)alkoxy, perhalo lower(C₁₋₄)alkyl or perhalo lower(C₁₋₄)alkoxy five to seven membered heterocyclic ring containing one to four heteroatoms selected from the group consisting of oxygen, nitrogen and sulphur;
R₁ and R₂ are independently selected from the group consisting of hydrogen, straight chain or branched alkyl groups having 1 to 3 carbon atoms selected from the group consisting of methyl, ethyl, propyl and isopropyl;
Y is CH or N;
Z is selected from the group consisting of 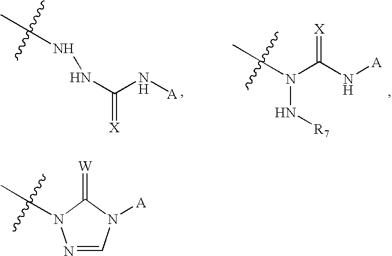
wherein
X is selected from S, O, CH—NO₂, and N—CN;
W is selected from S, CH—NO₂, and N—CN;
A is hydrogen, unsubstituted or substituted lower (C₁₋₁₀)alkyl, said substituents being halogen (fluorine, chlorine, bromine, iodine), hydroxy, lower (C₁₋₄)alkoxy, lower (C₁₋₄)perhaloalkyl, lower (C₁₋₄)perhaloalkoxy; optionally substituted naphthyl; unsubstituted or substituted aromatic or non aromatic 5-6 membered rings with or without one to four heteroatoms independently selected from the group consisting of oxygen, nitrogen and sulphur, said substituents independently selected from one or more groups such as halogen (fluorine, chlorine, bromine, iodine), nitro, cyano, hydroxy, lower (C₁₋₄) alkyl, lower(C₁₋₄)alkoxy, lower (C₁₋₄)perhaloalkyl, lower (C₁₋₄)perhaloalkoxy, BR₃, substituted or unsubstituted five or six membered heterocyclic ring systems containing one to four heteroatoms selected from the group consisting of oxygen, nitrogen and sulphur, said heterocycylic substituents being (C₁-C₈)alkanoyl, lower (C₁-C₄)alkyl, lower (C₁-C₄)alkoxy carbonyl, N lower (C₁-C₄)alkylaminocarbonyl, N,N-dilower(C₁-C₄)alkylaminocarbonyl, N-lower (C₁-C₄)alkylaminothiocarbonyl, N,N-di(lower alkyl)(C₁-C₄)aminothiocarbonyl, N-lower (C₁-C₄)alkyl sulphonyl, phenyl substituted lower (C₁-C₄)alkyl sulphonyl, N-lower (C₁-C₄)alkyl amino, N,N-di(lower alkyl)(C₁-C₄)amino, unsubstituted or substituted phenyl, said substituents being halogen (fluorine, chlorine, bromine, iodine), hydroxy, lower (C₁₋₄)alkoxy, lower (C₁₋₄)perhaloalkyl, lower (C₁₋₄)perhaloalkoxy, nitro, cyano, amino, N(R₄)₂, 5-6 membered heterocyclic rings, the preferred heterocycles being 1,3-imidazolyl; 1,2,4 triazolyl; —CHR₅R₆;
wherein
R₃ is a five or six membered aromatic or non aromatic ring with or without heteroatoms selected from the group consisting of oxygen, nitrogen and sulphur;
B is independently selected from (CH₂)ₘ, —S, —O(CH₂)ₘ, —S(CH₂)ₘ;
m is an integer from 1 to 4;
R₄ is hydrogen, unsubstituted or substituted lower (C₁₋₄)alkyl;
R₅ is —COQ, where Q=OR₄, —N(R₄)₂;
R₆ is independently selected from hydrogen, straight chain or branched alkyl with or without substituents, the said substituents being halogen (e.g. fluorine, chlorine, bromine or iodine), hydroxy, lower (C₁₋₄)alkyl, lower (C₁₋₄)alkoxy, lower (C₁₋₄)perhaloalkyl, lower (C₁₋₄)perhaloalkoxy, SR₄; phenyl or phenyl substituted with halogen (fluorine, chlorine, bromine, iodine), hydroxy, lower (C₁₋₄)alkoxy, lower (C₁₋₄)perhaloalkyl, lower (C₁₋₄)perhaloalkoxy, SR₄; heterocyclic rings or substituted heterocyclic rings with heteroatoms selected from oxygen, nitrogen and sulphur, substituents on heterocyclic rings are independently selected from halogen (fluorine, chlorine, bromine, iodine), hydroxy, lower (C₁₋₄)alkyl, lower (C₁₋₄)alkoxy, lower (C₁₋₄)perhaloalkyl, lower (C₁₋₄)perhaloalkoxy, SR₄; phenyl or phenyl substituted with halogen (e.g. fluorine, chlorine, bromine or iodine), hydroxy, lower (C₁₋₄)alkoxy, lower (C₁₋₄)perhaloalkyl, lower (C₁₋₄)perhaloalkoxy or SR₄; the preferred heterocyclic rings are imidazole and indole;
R₇ is H or selected from the group consisting of 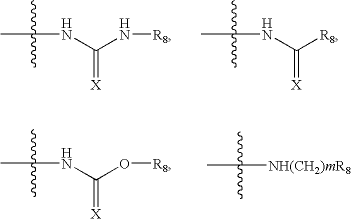
wherein
R₈ is independently selected from hydrogen, unsubstituted or substituted lower (C₁₋₄)alkyl, aralkyl, aromatic or non aromatic 5-6 membered rings with or without one to four heteroatoms selected independently from the group consisting of oxygen, nitrogen or sulphur.

2. A compound selected from the group consisting of:
1-t-Butoxycarbonyl-2-[(1R,2R)-2-(2,4-difluorophenyl)-2-hydroxy-1-methyl-3-(1H-1,2,4-triazol-1-yl)propyl]-4-[4-fluorophenyl]thiosemicarbazide
1-t-Butoxycarbonyl-2-[(1R,2R)-2-(2,4-difluorophenyl)-2-hydroxy-1-methyl-3-(1H-1,2,4-triazol-1-yl)propyl]4-[2,4-difluorophenyl]thiosemicarbazide
1-t-Butoxycarbonyl-2-[(1R,2R)-2-(2,4-difluorophenyl)-2-hydroxy-1-methyl-3-(1H-1,2,4-triazol-1-yl)propyl]-4-[4-trifluoromethylphenyl]thiosemicarbazide
1-t-Butoxycarbonyl-2-[(1R,2R)-2-(2,4-difluorophenyl)-2-hydroxy-1-methyl-3-(1H-1,2,4-triazol-1-yl)propyl]-4-[2,4-dimethoxyphenyl]thiosemicarbazide
1-t-Butoxycarbonyl-2-[(1R,2R)-2-(2,4-difluorophenyl)-2-hydroxy-1-methyl-3-(1H-1,2,4-triazol-1-yl)propyl]-4-[4(tetrahydropyranyloxy)phenyl]thiosemicarbazide
1-t-Butoxycarbonyl-2-[(1R,2R)-2-(2,4-difluorophenyl)-2-hydroxy-1-methyl-3-(1H-1,2,4-triazol-1-yl)propyl]-4-[4-trifluoromethoxyphenyl]thiosemicarbazide 1-t-Butoxycarbonyl-2-[(1R,2R)-2-(2,4-difluorophenyl)-2-hydroxy-1-methyl-3-(1H-1,2,4-triazol-1-yl)propyl]-4-[4-(2,2,3,3-tetrafluoropropoxy)phenyl]thiosemicarbazide
1-t-Butoxycarbonyl-2-[(1R,2R)-2-(2,4-difluorophenyl)-2-hydroxy-1-methyl-3-(1H-1,2,4-triazol-1-yl)propyl]-4-[4-nitrophenyl]thiosemicarbazide
1-t-Butoxycarbonyl-2-[(1R,2R)-2-(2,4-difluorophenyl)-2-hydroxy-1-methyl-3-(1H-1,2,4-triazol-1-yl)propyl]-4-[4-([1,2,3,4-tetrazol-1-yl])phenyl]thiosemicarbazide
1-t-Butoxycarbonyl-2-[(1R,2R)-2-(2,4-difluorophenyl)-2-hydroxy-1-methyl-3-(1H-1,2,4-triazol-1-yl)propyl]-4-[4-(1,2,3,4-tetrazol-2-yl)phenyl]thiosemicarbazide
1-t-Butoxycarbonyl-2-[(1R,2R)-2-(2,4-difluorophenyl)-2-hydroxy-1-methyl-3-(1H-1,2,4-triazol-1-yl)propyl]-4-[4-cyanophenyl]thiosemicarbazide
1-t-Butoxycarbonyl-2-[(1R,2R)-2-(2,4-difluorophenyl)-2-hydroxy-1-methyl-3-(1H-1,2,4-triazol-1-yl)propyl]-4-[4-[4-chlorophenyl]piperizin-1-yl]phenyl]thiosemicarbazide
1-t-Butoxycarbonyl-2-[(1R,2R)-2-(2,4-difluorophenyl)-2-hydroxy-1-methyl-3-(5 (1H-1,2,4triazol-1-yl)propyl]-4-[4-(N,N-dimethylamino)phenyl]thiosemi-carbazide
1-t-Butoxycarbonyl-2-(1R,2R)-2-(2,4-difluorophenyl)-2-hydroxy-1-methyl-3-(1H-1,2,4-triazol-1-yl)propyl]-4-napth-1-yl thiosemicarbazide
1-t-Butoxycarbonyl-2-(1R,2R)-2-(2,4-difluorophenyl)-2-hydroxy-1-methyl-3-(1H-1,2,4-triazol-1-yl)propyl]-4-octylthiosemicarbazide
1-t-Butoxycarbonyl-2-[(1R,2R)-2-(2,4difluorophenyl)-2-hydroxy-1-methyl-3-(1H-1,2,4-triazol-1-yl)propyl]-4-t-butyl thiosemicarbazide
Methyl-2-[1-t-butoxycarbonyl-2-[(1R,2R)-2-(2,4-difluorophenyl)-2-hydroxy-1-methyl-3-(1H-1,2,4-triazol-1-yl)propyl]thiosemicarbazid-4-yl]acetate
Methyl-2-phenyl-2-[1-t-butoxycarbonyl-2-[(1R,2R)-2-(2,4difluorophenyl)-2-hydroxy-1-methyl-3-(1H-1,2,4-triazol-1-yl)propyl]thiosemicarbazid-4-yl]acetate
Methyl-2-[t-butyldimethylsilyloxymethyl]-2-[1-t-butoxycarbonyl-2-[(1R,2R)-2-(2,4-difluorophenyl)-2-hydroxy-1-methyl-3-(1H-1,2,4-triazol-1-yl)propyl]thiosemicarbazid4-yl]acetate
Methyl-2-[methylthioethyl]-2-[1-t-butoxycarbonyl-2-[(1R,2R)-2-(2,4-difluorophenyl)-2-hydroxy-1-methyl-3-(1H-1,2,4-triazol-1-yl)propyl]thiosemicarbazid-4-yl]acetate
Methyl-2-benzyl-2-[1-t-butoxycarbonyl-2-[(1R,2R)-2-(2,4-difluorophenyl)-2-hydroxy-1-methyl-3-(1H-1,2,4-triazol-1-yl)propyl]thiosemicarbazid-4-yl]acetate
Methyl-2-isobutyl-2-[1-t-butoxycarbonyl-2-[(1R,2R)-2-(2,4-difluorophenyl)-2-hydroxy-1-methyl-3-(1H-1,2,4-triazol-1-yl)propyl]thiosemicarbazid-4-yl]acetate
1-t-Butoxycarbonyl-2-[(1R,2R)-2-(2,4-difluorophenyl)-2-hydroxy-1-methyl-3-(1H-1,2,4-triazol-1-yl)propyl]-4-[2-furanmethyl]thiosemicarbazide
1-t-Butoxycarbonyl-2-[(1R,2R)-2-(2,4-difluorophenyl)-2-hydroxy-1-methyl-3-(1H-1,2,4-triazol-1-yl)propyl]-4-[2-thiophenmethyl]thiosemicarbazide
1-t-Butoxycarbonyl-2-[(1R,2R)-2-(2,4-difluorophenyl)-2-hydroxy-1-methyl-3-(1H-1,2,4-triazol-1-yl)propyl]-4-[4-chlorophenyl]semicarbazide
1-t-Butoxycarbonyl-2-[(1R,2R)-2-(2,4-difluorophenyl)-2-hydroxy-1-methyl-3-(1H-1,2,4-triazol-1-yl)propyl]-4-[4(2,2,3,3-tetrafluoropropoxy)phenyl]semicarbazide
1-t-Butoxycarbonyl-2-[(1R,2R)-2-(2,4-difluorophenyl)-2-hydroxy-1-methyl-3-(1H-1,2,4-triazol-1-yl)propyl]-4-[2,4-dimethoxyphenyl]semicarbazide
2-[(1R,2R)-2-(2,4-difluorophenyl)-2-hydroxy-1-methyl-3-(1H-1,2,4-triazol-1-yl)propyl]-4-[4-chlorophenyl]semicarbazide
2-[(1R,2R)-2-(2,4-difluorophenyl)-2-hydroxy-1-methyl-3-(1H-1,2,4-triazol-1-yl)propyl]-4-phenyl thiosemicarbazide
2-[(1R,2R)-2-(2,4-difluorophenyl)-2-hydroxy-1-methyl-3-(1H-1,2,4-triazol-1-yl)propyl]-4-[4-hydroxyphenyl]thiosemicarbazide
2-[(1R,2R)-2-(2,4-difluorophenyl)-2-hydroxy-1-methyl-3-(1H-1,2,4-triazol-1-yl)propyl]-4-[4-(2,2,3,3-tetrafluoropropoxy)phenyl]thiosemicarbazide
2-[(1R,2R)-2-(2,4-difluorophenyl)-2-hydroxy-1-methyl-3-(1H-1,2,4-triazol-1-yl)propyl]-4-[2,4-dimethoxyphenyl]thiosemicarbazide
2-[(1R,2R)-2-(2,4-difluorophenyl)-2-hydroxy-1-methyl-3-(1H-1,2,4-triazol-1-yl)propyl]-4-[4-trifluoromethylphenyl]thiosemicarbazide
2-[(1R,2R)-2-(2,4-Difluorophenyl)-2-hydroxy-1-methyl-3-(1H-1,2,4-triazol-1-yl)propyl]-4-[4-trifluoromethoxyphenyl]thiosemicarbazide
2-[(1R,2R)-2-(2,4-difluorophenyl)-2-hydroxy-1-methyl-3-(1H-1,2,4-triazol-1-yl)propyl]-4-[2-furanmethyl]thiosemicarbazide
2-[(1R,2R)-2-(2,4-difluorophenyl)-2-hydroxy-1-methyl-3-(1H-1,2,4-triazol-1-yl)propyl]-4-[2-thiophenmethyl]thiosemicarbazide
2-[(1R,2R)-2-(2,4-Difluorophenyl)-2-hydroxy-1-methyl-3-(1H-1,2,4-triazol-1-yl)propyl]-4-[3-chloropyridin-6-yl]thiosemicarbazide
2-[(1R,2R)-2-(2,4-Difluorophenyl)-2-hydroxy-1-methyl-3-(1H-1,2,4-triazol-1-yl)propyl]-4-[5-chloro-3-trifluromethyl-pyridin-6-yl]thiosemicarbazide
2-[(1R,2R)-2-(2,4-Difluorophenyl)-2-hydroxy-1-methyl-3-(1H-1,2,4-triazol-1-yl)propyl]-4-[quinolin-3-yl]thiosemicarbazide
2-[(1R,2R)-2-(2,4-difluorophenyl)-2-hydroxy-1-methyl-3-(1,2,4-triazol-1-yl)propyl]-4-[4-(1,2,3,4-tetrazol-1-yl)phenyl]-(2H,4H)-1,2,4-triazol-3-thione
2-[(1R,2R)-2-(2,4-difluorophenyl)-2-hydroxy-1-methyl-3-(1,2,4-triazol-1-yl)propyl]-4-[4-hydroxyphenyl]-(2H,4H)-1,2,4-triazol-3-thione
2-[(1R,2R)-2-(2,4difluorophenyl)-2-hydroxy-1-methyl-3-(1,2,4-triazol-1-yl)propyl]-4-[4-(2,2,3,3-tetrafluoropropoxy)phenyl]-(2H,4H)-1,2,4-triazol-3-thione
2-[(1R,2R)-2-(2,4-difluorophenyl)-2-hydroxy-1-methyl-(1,2,4-triazol-1-yl)propyl]-4-[4-nitrophenyl]-(2H,4H)-1,2,4-triazol-3-thione
2-[(1R,2R)-2-(2,4-difluorophenyl)-2-hydroxy-1-methyl-3-(1,2,4-triazol-1-yl)propyl]-4[4-(1,2,3,4-tetrazol-2-yl))phenyl]-(2H,4H)-1,2,4-triazol-3-thione
2-[(1R,2R)-2-(2,4difluorophenyl)-2-hydroxy-1-methyl-3-(1,2,4-triazol-1-yl)propyl]-4-[4-trifluoromethylphenyl]-(2H,4H)-1,2,4-triazol-3-thione
2-[(1R,2R)-2-(2,4Difluorophenyl)-2-hydroxy-1-methyl-3(1H-1,2,4-triazol-1-yl)propyl]-4-[4-trifluoromethoxyphenyl]-(2H,4H)-1,2,4-triazol-3-thione
2-[(1R,2R)-2-(2,4-difluorophenyl)-2-hydroxy-1-methyl-3-(1,2,4-triazol-1-yl)propyl]-4-[4-cyanophenyl](2H,4H)-1,2,4-triazol-3-thione
Methyl-2-[[(1R,2R)-2-(2,4-difluorophenyl)-2-hydroxy-1-methyl-3-(1H-1,2,4-triazol-1-yl)propyl]-(2H,4H)-1,2,4-triazol-3-thion-4-yl]acetate
Methyl-2-hydroxymethyl-2-[[(1R,2R)-2-(2,4-difluorophenyl)-2-hydroxy-1-methyl-3-(1H-1,2,4-triaolyl)propyl]-(2H,4H)-1,2,4-triazol-3-thion-4-yl]acetate
Methyl-2-phenyl-2-[[(1R,2R)-2-(2,4-difluorophenyl)-2-hydroxy-1-methyl-3-(1H-1,2,4-triaolyl)propyl]-(2H,4H)-1,2,4-triazol-3-thion-4-yl]acetate
Methyl-2-isobutyl-2-[[(1R,2R)-2-(2,4-difluorophenyl)-2-hydroxy-1-methyl-3-(1H-1,2,4-triaolyl)propyl]-(2H,4H)-1,2,4-triazol-3-thion-4-yl]acetate
Methyl-2-methylthioethyl-2-[[(1R,2R)-2-(2,4-difluorophenyl)-2-hydroxy-1-methyl-3-(1H-1,2,4-triaolyl)propyl]-(2H,4H)-1,2,4-triazol-3-thion-4-yl]acetate
2-[(1R,2R)-2-(2,4-Difluorophenyl)-2-hydroxy-1-methyl-3-(1H-1,2,4-triazol-1-yl)propyl]-4-[2-furanmethyl]-(2H,4H)-1,2,4triazol-3-thione
2-[(1R,2R)-2-(2,4-Difluorophenyl)-2-hydroxy-1-methyl-3-(1H-1,2,4-triazol-1-yl)propyl]-4-[quinolin-3-yl]-(2H,4H)-1,2,4-triazol-3-thione
2-[(1R,2R)-2-(2,4-Difluorophenyl)-2-hydroxy-1-methyl-3-(1H-1,2,4-triazol-1-yl)propyl]-4-[3-chloropyridin-6-yl]-(2H,4H)-1,2,4-triazol-3-thione
2-[(1R,2R)-2-(2,4-Difluorophenyl)-2-hydroxy-1-methyl-3-(1H-1,2,4-triazol-1-yl)propyl]-4-[5-chloro-3-trifluoromethylpyridin-6-yl]-(2H,4H)-1,2,4-triazol-3-thione

3. A pharmaceutical composition comprising a compound of claims 1 or 2 and a pharmaceutical acceptable carrier.

4. A pharmaceutical composition comprising a pharmaceutically effective amount of a compound according to claims 1 to 3 or a physiologially acceptable acid additional salt thereof with a pharmaceuitcally acceptable carrier.

5. A method of treating or preventing fungal infection in mammals comprising administering to said mammal a therapeutically effective amount of a compound having the structure of Formula I, 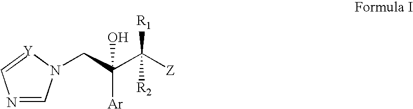
and its pharmaceutically acceptable salts, esters, enantiomers, diastereomers, N-oxides, prodrugs, metabolites, polymorphs or pharmaceutically acceptable solvates,
wherein
Ar is phenyl or a substituted phenyl having one to three substituents independently selected from halogen (chlorine, fluorine, bromine, iodine), nitro, cyano, lower(C₁₋₄)alkyl, lower(C₁₋₄)alkoxy, perhalo lower(C₁₋₄)alkyl or perhalo lower(C₁₋₄)alkoxy five to seven membered heterocyclic ring containing one to four heteroatoms selected from the group consisting of oxygen, nitrogen and sulphur;
R₁ and R₂ are independently selected from the group consisting of hydrogen, straight chain or branched alkyl groups having 1 to 3 carbon atoms selected from the group consisting of methyl, ethyl, propyl and isopropyl;
Y is CH or N;
Z is selected from the group consisting of 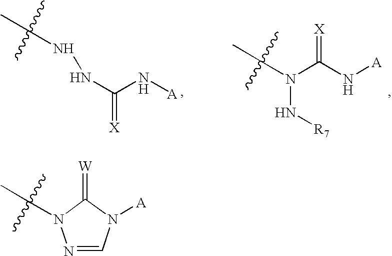
wherein
X is selected from S, O, CH—NO₂, N—CN;
W is selected from S, CH—NO₂, N—CN;
A is hydrogen, unsubstituted or substituted lower (C₁₋₁₀)alkyl, said substituents being halogen (fluorine, chlorine, bromine, iodine), hydroxy, lower (C₁₋₄)alkoxy, lower (C₁₋₄)perhaloalkyl, lower (C₁₋₄)perhaloalkoxy; optionally substituted naphthyl; unsubstituted or substituted aromatic or non aromatic 5-6 membered rings with or without one to four heteroatoms independently selected from the group consisting of oxygen, nitrogen and sulphur, said substituents independently selected from one or more groups such as halogen (fluorine, chlorine, bromine, iodine), nitro, cyano, hydroxy, lower(C₁₋₄)alkyl, lower(C₁₋₄)alkoxy, lower (C₁₋₄)perhaloalkyl, lower (C₁₋₄)perhaloalkoxy, BR₃, substituted or unsubstituted five or six membered heterocyclic ring systems containing one to four heteroatoms selected from the group consisting of oxygen, nitrogen and sulphur, said heterocycylic substituents being (C₁-C₈)alkanoyl, lower (C₁-C₄)alkyl, lower (C₁-C₄)alkoxy carbonyl, N lower (C₁-C₄)alkylaminocarbonyl, N,N-dilower(C₁-C₄)alkylaminocarbonyl, N-lower (C₁-C₄)alkylaminothiocarbonyl, N,N-di(lower alkyl)(C₁-C₄)aminothiocarbonyl, N-lower (C₁-C₄)alkyl sulphonyl, phenyl substituted lower (C₁-C₄)alkyl sulphonyl, N-lower (C₁-C₄)alkyl amino, N,N-di(lower alkyl)(C₁-C₄)amino, unsubstituted or substituted phenyl, said substituents being halogen (fluorine, chlorine, bromine, iodine), hydroxy, lower (C₁₋₄)alkoxy, lower (C₁₋₄)perhaloalkyl, lower (C₁₋₄)perhaloalkoxy, nitro, cyano, amino, N(R₄)₂, 5-6 membered heterocyclic rings, the preferred heterocycles being 1,3-imidazolyl; 1,2,4 triazolyl; —CHR₅R₆;
wherein
R₃ is a five or six membered aromatic or non aromatic ring with or without heteroatoms selected from (oxygen, nitrogen and sulphur);
B is independently selected from (CH₂)ₘ, —S, —O(CH₂)ₘ, —S(CH₂)ₘ;
m is an integer from 1 to 4;
R₄ is hydrogen, unsubstituted or substituted lower (C₁₋₄)alkyl;
R₅ is —COQ, where Q=OR₄, —N(R₄)₂;
R₆ is independently selected from hydrogen, straight chain or branched alkyl with or without substituents, said substituents being halogen (e.g. fluorine, chlorine, bromine or iodine), hydroxy, lower (C₁₋₄)alkyl, lower (C₁₋₄)alkoxy, lower (C₁₋₄)perhaloalkyl, lower (C₁₋₄)perhaloalkoxy, SR₄; phenyl or phenyl substituted with halogen (fluorine, chlorine, bromine, iodine), hydroxy, lower (C₁₋₄)alkoxy, lower (C₁₋₄)perhaloalkyl, lower (C₁₋₄)perhaloalkoxy, SR₄; heterocyclic rings or substituted heterocyclic rings with heteroatoms selected from oxygen, nitrogen and sulphur, substituents on heterocyclic rings are independently selected from halogen (fluorine, chlorine, bromine, iodine), hydroxy, lower (C₁₋₄)alkyl, lower (C₁₋₄)alkoxy, lower (C₁₋₄)perhaloalkyl, lower (C₁₋₄)perhaloalkoxy, SR₄; phenyl or phenyl substituted with halogen (e.g. fluorine, chlorine, bromine or iodine), hydroxy, lower (C₁₋₄)alkoxy, lower (C₁₋₄)perhaloalkyl, lower (C₁₋₄)perhaloalkoxy or SR₄; the preferred heterocyclic rings are imidazole and indole;
R₇ is H or selected from the group consisting of 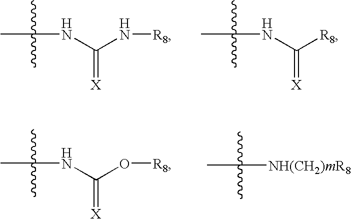
wherein
R₈ is independently selected from hydrogen, unsubstituted or substituted lower (C₁₋₄)alkyl, aralkyl, aromatic or non aromatic 5-6 membered rings with or without one to four heteroatoms selected independently from the group consisting of oxygen, nitrogen or sulphur.

6. A process for preparing a compound of Formula X, 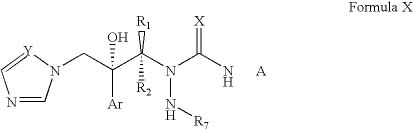
and its pharmaceutically acceptable salts, esters, enantiomers, diastereomers, N-oxides, prodrugs, metabolites, polymorphs or pharmaceutically acceptable solvates
wherein
Ar is phenyl or a substituted phenyl having one to three substituents independently selected from halogen (chlorine, fluorine, bromine, iodine), nitro, cyano, lower(C₁₋₄)alkyl, lower(C₁₋₄)alkoxy, perhalo lower(C₁₋₄)alkyl or perhalo lower(C₁₋₄)alkoxy five to seven membered heterocyclic ring containing one to four heteroatoms selected from the group consisting of oxygen, nitrogen and sulphur;
R₁ and R₂ are independently selected from the group consisting of hydrogen, straight chain or branched alkyl groups having 1 to 3 carbon atoms selected from the group consisting of methyl, ethyl, propyl and isopropyl;
Y is CH or N;
X is selected from S, O, CH—NO₂, N—CN;
A is hydrogen, unsubstituted or substituted lower (C₁₋₁₀)alkyl, said substituents being halogen (fluorine, chlorine, bromine, iodine), hydroxy, lower (C₁₋₄)alkoxy, lower (C₁₋₄)perhaloalkyl, lower (C₁₋₄)perhaloalkoxy; optionally substituted naphthyl; unsubstituted or substituted aromatic or non aromatic 5-6 membered rings with or without one to four heteroatoms independently selected from the group consisting of oxygen, nitrogen and sulphur, said substituents independently selected from one or more groups such as halogen (fluorine, chlorine, bromine, iodine), nitro, cyano, hydroxy, lower(C₁₋₄)alkyl, lower(C₁₋₄)alkoxy, lower (C₁₋₄)perhaloalkyl, lower (C₁₋₄l)perhaloalkoxy, BR₃, substituted or unsubstituted five or six membered heterocyclic ring systems containing one to four heteroatoms selected from the group consisting of oxygen, nitrogen and sulphur, said heterocycylic substituents being (C₁-C₈)alkanoyl, lower (C₁-C₄)alkyl, lower (C₁-C₄)alkoxy carbonyl, N lower (C₁-C₄)alkylaminocarbonyl, N,N-dilower(C₁-C₄)alkylaminocarbonyl, N-lower (C₁-C₄)alkylaminothiocarbonyl, N,N-di(lower alkyl)(C₁-C₄)aminothiocarbonyl, N-lower (C₁-C₄)alkyl sulphonyl, phenyl substituted lower (C₁-C₄)alkyl sulphonyl, N-lower (C₁-C₄)alkyl amino, N,N-di(lower alkyl)(C₁-C₄)amino, unsubstituted or substituted phenyl, the said substituents being halogen (fluorine, chlorine, bromine, iodine), hydroxy, lower (C₁₋₄)alkoxy, lower (C₁₋₄)perhaloalkyl, lower (C₁₋₄)perhaloalkoxy, niro, cyano, amino, N(R₄)₂, 5-6 membered heterocyclic rings, the preferred heterocycles being 1,3-imidazolyl; 1,2,4 triazolyl; —CHR₅R₆;
wherein
R₃ is a five or six membered aromatic or non aromatic ring with or without heteroatoms selected from (oxygen, nitrogen and sulphur);
B is independently selected from (CH₂)ₘ, —S, —O(CH₂)ₘ, —S(CH₂)ₘ;
m is an integer from 1 to 4;
R₄ is hydrogen, unsubstituted or substituted lower (C₁₋₄)alkyl;
R₅ is —COQ, where Q=OR₄, —N(R₄)₂;
R₆ is independently selected from hydrogen, straight chain or branched alkyl with or without substituents, said substituents being halogen (e.g. fluorine, chlorine, bromine or iodine), hydroxy, lower (C₁₋₄)alkyl, lower (C₁₋₄)alkoxy, lower (C₁₋₄)perhaloalkyl, lower (C₁₋₄)perhaloalkoxy, SR₄; phenyl or phenyl substituted with halogen (fluorine, chlorine, bromine, iodine), hydroxy, lower (C₁₋₄)alkoxy, lower (C₁₋₄)perhaloalkyl, lower (C₁₋₄)perhaloalkoxy, SR₄; heterocyclic rings or substituted heterocyclic rings with heteroatoms selected from oxygen, nitrogen and sulphur, substituents on heterocyclic rings are independently selected from halogen (fluorine, chlorine, bromine, iodine), hydroxy, lower (C₁₋₄)alkyl lower (C₁₋₄)alkoxy, lower (C₁₋₄)perhaloalkyl, lower (C₁₋₄)perhaloalkoxy, SR₄; phenyl or phenyl substituted with halogen (e.g. fluorine, chlorine, bromine or iodine), hydroxy, lower (C₁₋₄)alkoxy, lower (C₁₋₄)perhaloalkyl, lower (C₁₋₄)perhaloalkoxy or SR₄; the preferred heterocyclic rings are imidazole and indole;
R₇ is H or selected from the group consisting of 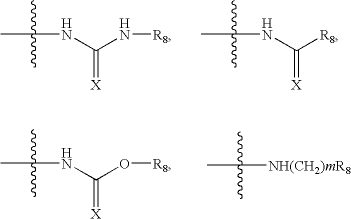
wherein
R₈ is independently selected from hydrogen, unsubstituted or substituted lower (C₁₋₄)alkyl, aralkyl, aromatic or non aromatic 5-6 membered rings with or without one to four heteroatoms selected independently from the group consisting of oxygen, nitrogen or sulphur,
which comprises converting the epoxy alcohol of Formula II 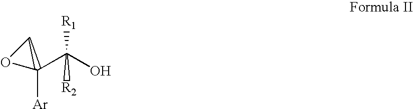
to the corresponding triflate derivative, which is further subjected to a nucleophilic substitution with t-butyl carbazate to afford substituted hydrazine of the Formula III 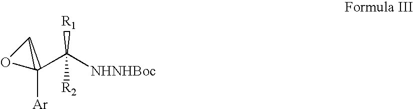
with inversion of configuration at C-1, which on reaction with compound of Formula IV, 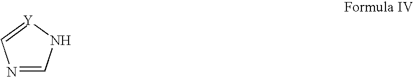
in the presence of a base gives the epoxide ring opened intermediate of the formula V, 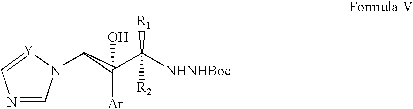
which is then treated with the compound of the Formula VI
A-N═C═X Formula VI
to give the BoC protected semicarbazide or thiosemicarbazide derivatives of the Formula VII, 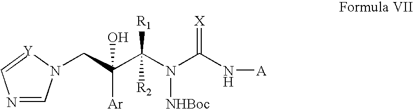
which is further deprotected using trifluoroacetic acid to give the free amine of Formula VIII, 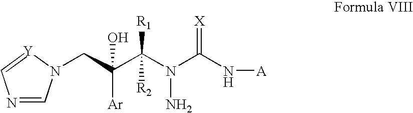
which is treated with a compound of Formula IX
R₇Cl Formula IX
to give a compound of Formula X.

7. The process of claim 6 wherein the conversion of the compound of Formula II to the compound of Formula III is carried out in an organic solvent selected from the group consisting of chloroform, dichloromethane, dichloroethane and tetrahydrofuran.

8. The process of claim 6 wherein the nucleophilic epoxide ring opening of the compound of Formula IV is carried out in the presence of a base selected from the group consisting of potassium carbonate, cesium carbonate, calcium carbonate and sodium hydride.

9. The process according to claim 6 wherein the nucleophilic epoxide ring opening of the compound of Formula IV is carried out in a solvent selected from the group consisting of dimethylformamide, dimethylsulfoxide, diethyl ether, tetrahydrofuran, toluene, benzene and mixtures thereof.

10. The process according to claim 6 wherein the reaction of the compound of Formula V with a compound of Formula VI to give a compound of Formula VII is carried out in an organic solvent selected from the group consisting of chloroform, dichloromethane, dichloroethane, and tetrahydrofuran and mixtures thereof.

11. The process according to claim 6 wherein the deprotection of the Boc group in the compound of Formula VII to give the free amine of Formula VIII is carried out in an organic solvent selected from the group consisting of chloroform, dichloromethane, dichloroethane, tetrahydrofuran and mixtures thereof.

12. The process according to claim 6 wherein the reaction of the compound of Formula VIII with a compound of Formula IX to give a compound of Formula X is carried out in an organic solvent selected from the group consisting of chloroform, dichloromethane, dichloroethane, tetrahydrofuran and mixtures thereof.

13. The process according to claim 6 wherein the reaction of the compound of Formula V with the isothiocyanate of Formula XI is carried out in an organic solvent selected from the group consisting of chloroform, dichloromethane, dichloroethane, tetrahydrofuran and mixtures thereof.

14. A process for preparing a compound of Formula XIII, 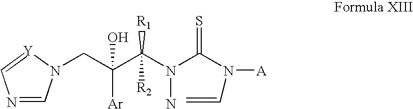
and its pharmaceuitcally acceptable salts, esters, enantiomers, diastereomers, N-oxides, prodrugs, metabolites, polymorphs and pharmaceutically acceptable solvates,
wherein
Ar is phenyl or a substituted phenyl having one to three substituents independently selected from halogen (chlorine, fluorine, bromine, iodine), nitro, cyano, lower(C₁₋₄)alkyl, lower(C₁₋₄)alkoxy, perhalo lower(C₁₋₄)alkyl or perhalo lower(C₁₋₄)alkoxy five to seven membered heterocyclic ring containing one to four heteroatoms selected from the group consisting of oxygen, nitrogen and sulphur;
R₁ and R₂ are independently selected from the group consisting of hydrogen, straight chain or branched alkyl groups having 1 to 3 carbon atoms selected from the group consisting of methyl, ethyl, propyl and isopropyl;
Y is CH or N;
A is hydrogen, unsubstituted or substituted lower (C₁₋₁₀)alkyl, said substituents being halogen (fluorine, chlorine, bromine, iodine), hydroxy, lower (C₁₋₄)alkoxy, lower (C₁₋₄)perhaloalkyl, lower (C₁₋₄)perhaloalkoxy; optionally substituted naphthyl; unsubstituted or substituted aromatic or non aromatic 5-6 membered rings with or without one to four heteroatoms independently selected from the group consisting of oxygen, nitrogen and sulphur, the said substituents independently selected from one or more groups such as halogen (fluorine, chlorine, bromine, iodine), nitro, cyano, hydroxy, lower(C₁₋₄)alkyl, lower(C₁₋₄)alkoxy, lower (C₁₋₄)perhaloalkyl, lower (C₁₋₄)perhaloalkoxy, BR₃, substituted or unsubstituted five or six membered heterocyclic ring systems containing one to four heteroatoms selected from the group consisting of oxygen, nitrogen and sulphur, said heterocycylic substituents being (C₁-C₈)alkanoyl, lower (C₁-C₄)alkyl, lower (C₁-C₄)alkoxy carbonyl, N lower (C₁-C₄)alkylaminocarbonyl, N,N-dilower(C₁-C₄)alkylaminocarbonyl, N-lower (C₁-C₄)alkylaminothiocarbonyl, N,N-di(lower alkyl)(C₁-C₄)aminothiocarbonyl, N-lower (C₁-C₄)alkyl sulphonyl, phenyl substituted lower (C₁-C₄)alkyl sulphonyl, N-lower (C₁-C₄)alkyl amino, N,N-di(lower alkyl)(C₁-C₄)amino, unsubstituted or substituted phenyl, the said substituents being halogen (fluorine, chlorine, bromine, iodine), hydroxy, lower (C₁₋₄)alkoxy, lower (C₁₋₄)perhaloalkyl, lower (C₁₋₄)perhaloalkoxy, niro, cyano, amino, N(R₄)₂, 5-6 membered heterocyclic rings, the preferred heterocycles being 1,3-imidazolyl; 1,2,4 triazolyl; —CHR₅R₆;
wherein
R₃ is a five or six membered aromatic or non aromatic ring with or without heteroatoms selected from (oxygen, nitrogen and sulphur);
B is independently selected from (CH₂)ₘ, —S, —O(CH₂)ₘ, —S(CH₂)ₘ;
m is an integer from 1 to 4;
R₄ is hydrogen, unsubstituted or substituted lower (C₁₋₄)alkyl;
R₅ is —COQ, where Q=OR₄, —N(R₄)₂;
R₆ is independently selected from hydrogen, straight chain or branched alkyl with or without substituents, said substituents being halogen (e.g. fluorine, chlorine, bromine or iodine), hydroxy, lower (C₁₋₄)alkyl, lower (C₁₋₄)alkoxy, lower (C₁₋₄)perhaloalkyl, lower (C₁₋₄)perhaloalkoxy, SR₄; phenyl or phenyl substituted with halogen (fluorine, chlorine, bromine, iodine), hydroxy, lower (C₁₋₄)alkoxy, lower (C₁₋₄)perhaloalkyl, lower (C₁₋₄)perhaloalkoxy, SR₄; heterocyclic rings or substituted heterocyclic rings with heteroatoms selected from oxygen, nitrogen and sulphur, substituents on heterocyclic rings are independently selected from halogen (fluorine, chlorine, bromine, iodine), hydroxy, lower (C₁₋₄)alkyl, lower (C₁₋₄)alkoxy, lower (C₁₋₄)perhaloalkyl, lower (C₁₋₄)perhaloalkoxy, SR₄; phenyl or phenyl substituted with halogen (e.g. fluorine, chlorine, bromine or iodine), hydroxy, lower (C₁₋₄)alkoxy, lower (C₁₋₄)perhaloalkyl, lower (C₁₋₄)perhaloalkoxy or SR₄; the preferred heterocyclic rings are imidazole and indole,
which comprises treating the compound of formula V 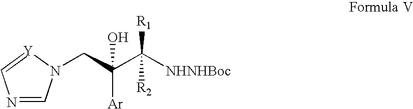
with the isothiocyanate of Formula XI
A-N═C═S Formula XI
and the resulting BoC derivatives of Formula XII 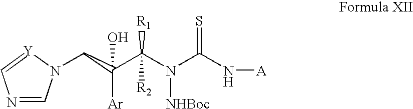
is refluxed with formic acid to give the desired compound of Formula XIII,
or alternatively, treating the compound of Formula XII with trifluoroacelic acid to get the free amine of Formula XIV, 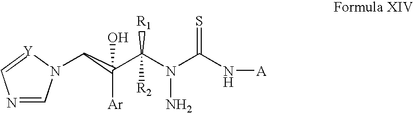
which upon refluxing with formic acid gives the compound of Formula XIII.

15. The process according to claim 14 wherein the reaction of the compound of Formula V with isothiocyanate of Formula XI is carried out in an organic solvent.

16. The process according to claim 15 wherein the organic solvent is selected from the group consisting of chloroform, dichloromethane, dichloroethane, tetrahydrofuran and mixtures thereof.

17. The process according to claim 14 wherein the deprotectioin of the BoC group in the compound of Formula XII to give the free amine of Formula XIV is carried out in an organic solvent.

18. The process according to claim 17 wherein the organic solvent is selected from the group consisting of chloroform, dichloromethane, dichloroethane, tetrahydrofuran and mixtures thereof.

19. The process according to claim 17 wherein the BoC deprotection of the compound of Formula XII is carried out in the presence of trifluroacetic acid (TFA).

20. The process according to claim 14 wherein the ring cyclization of the compound of Formula XII or its free amine of Formula XIV is carried out in the presence of formic acid.

21. The process according to claim 20 wherein the ring cyclization is carried out at a temperature ranging from about 80-120° C.

22. A compound having the structure of of Formula III 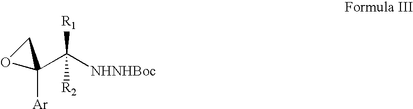
wherein Ar is phenyl or a substituted phenyl having one to three substituents independently selected from halogen (chlorine, fluorine, bromine, iodine), nitro, cyano, lower(C₁₋₄)alkyl, lower(C₁₋₄)alkoxy, perhalo lower(C₁₋₄)alkyl or perhalo lower(C₁₋₄)alkoxy five to seven membered heterocyclic ring containing one to four heteroatoms selected from the group consisting of oxygen, nitrogen and sulphur; and
R₁ and R₂ are independently selected from the group consisting of hydrogen, straight chain or branched alkyl groups having 1 to 3 carbon atoms selected from the group consisting of methyl, ethyl, propyl and isopropyl.

23. A compound having the structure of Formula V 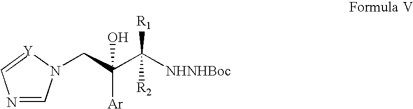
wherein Ar is phenyl or a substituted phenyl having one to three substituents independently selected from halogen (chlorine, fluorine, bromine, iodine), nitro, cyano, lower(C₁₋₄)alkyl, lower(C₁₋₄)alkoxy, perhalo lower(C₁₋₄)alkyl or perhalo lower(C₁₋₄)alkoxy five to seven membered heterocyclic ring containing one to four heteroatoms selected from the group consisting of oxygen, nitrogen and sulphur;
R₁ and R₂ are independently selected from the group consisting of hydrogen, straight chain or branched alkyl groups having 1 to 3 carbon atoms selected from the group consisting of methyl, ethyl, propyl and isopropyl; and
Y is CH or N.

24. A compound having the structure of Formula VII 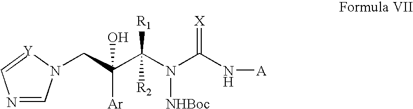
wherein Ar is phenyl or a substituted phenyl having one to three substituents independently selected from halogen (chlorine, fluorine, bromine, iodine), nitro, cyano, lower(C₁₋₄)alkyl, lower(C₁₋₄)alkoxy, perhalo lower(C₁₋₄)alkyl or perhalo lower(C₁₋₄)alkoxy five to seven membered heterocyclic ring containing one to four heteroatoms selected from the group consisting of oxygen, nitrogen and sulphur;
R₁ and R₂ are independently selected from the group consisting of hydrogen, straight chain or branched alkyl groups having 1 to 3 carbon atoms selected from the group consisting of methyl, ethyl, propyl and isopropyl;
Y is CH or N;
A is hydrogen, unsubstituted or substituted lower (C₁₋₁₀)alkyl, said substituents being halogen (fluorine, chlorine, bromine, iodine), hydroxy, lower (C₁₋₄)alkoxy, lower (C₁₋₄)perhaloalkyl, lower (C₁₋₄)perhaloalkoxy; optionally substituted naphthyl; unsubstituted or substituted aromatic or non aromatic 5-6 membered rings with or without one to four heteroatoms independently selected from the group consisting of oxygen, nitrogen and sulphur, said substituents independently selected from one or more groups such as halogen (fluorine, chlorine, bromine, iodine), nitro, cyano, hydroxy, lower (C₁₋₄) alkyl, lower(C₁₋₄)alkoxy, lower (C₁₋₄)perhaloalkyl, lower (C₁₋₄)perhaloalkoxy, BR₃, substituted or unsubstituted five or six membered heterocyclic ring systems containing one to four heteroatoms selected from the group consisting of oxygen, nitrogen and sulphur, said heterocycylic substituents being (C₁-C₈)alkanoyl, lower (C₁-C₄)alkyl, lower (C₁-C₄)alkoxy carbonyl, N lower (C₁-C₄)alkylaminocarbonyl, N,N-dilower(C₁-C₄)alkylaminocarbonyl, N-lower (C₁-C₄)alkylaminothiocarbonyl, N,N-di(lower alkyl)(C₁-C₄)aminothiocarbonyl, N-lower (C₁-C₄)alkyl sulphonyl, phenyl substituted lower (C₁-C₄)alkyl sulphonyl, N-lower (C₁-C₄)alkyl amino, N,N-di(lower alkyl)(C₁-C₄)amino, unsubstituted or substituted phenyl, said substituents being halogen (fluorine, chlorine, bromine, iodine), hydroxy, lower (C₁₋₄)alkoxy, lower (C₁₋₄)perhaloalkyl, lower (C₁₋₄)perhaloalkoxy, nitro, cyano, amino, N(R₄)₂, 5-6 membered heterocyclic rings, the preferred heterocycles being 1,3-imidazolyl; 1,2,4 triazolyl; —CHR₅R₆;
wherein
R₃ is a five or six membered aromatic or non aromatic ring with or without heteroatoms selected from the group consisting of oxygen, nitrogen and sulphur;
B is independently selected from (CH₂)ₘ, —S, —O(CH₂)ₘ, —S(CH₂)ₘ;
m is an integer from 1 to 4;
R₄ is hydrogen, unsubstituted or substituted lower (C₁₋₄)alkyl;
R₅ is —COQ, where Q=OR₄, —N(R₄)₂; and
R₆ is independently selected from hydrogen, straight chain or branched alkyl with or without substituents, the said substituents being halogen (e.g. fluorine, chlorine, bromine or iodine), hydroxy, lower (C₁₋₄)alkyl, lower (C₁₋₄)alkoxy, lower (C₁₋₄)perhaloalkyl, lower (C₁₋₄)perhaloalkoxy, SR₄; phenyl or phenyl substituted with halogen (fluorine, chlorine, bromine, iodine), hydroxy, lower (C₁₋₄)alkoxy, lower (C₁₋₄)perhaloalkyl, lower (C₁₋₄)perhaloalkoxy, SR₄; heterocyclic rings or substituted heterocyclic rings with heteroatoms selected from oxygen, nitrogen and sulphur, substituents on heterocyclic rings are independently selected from halogen (fluorine, chlorine, bromine, iodine), hydroxy, lower (C₁₋₄)alkyl, lower (C₁₋₄)alkoxy, lower (C₁₋₄)perhaloalkyl, lower (C₁₋₄)perhaloalkoxy, SR₄; phenyl or phenyl substituted with halogen (e.g. fluorine, chlorine, bromine or iodine), hydroxy, lower (C₁₋₄)alkoxy, lower (C₁₋₄)perhaloalkyl, lower (C₁₋₄)perhaloalkoxy or SR₄; the preferred heterocyclic rings are imidazole and indole.

25. A compound having the structure of Formula VIII 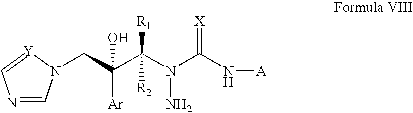
wherein Ar is phenyl or a substituted phenyl having one to three substituents independently selected from halogen (chlorine, fluorine, bromine, iodine), nitro, cyano, lower(C₁₋₄)alkyl, lower(C₁₋₄)alkoxy, perhalo lower(C₁₋₄)alkyl or perhalo lower(C₁₋₄)alkoxy five to seven membered heterocyclic ring containing one to four heteroatoms selected from the group consisting of oxygen, nitrogen and sulphur;
R₁ and R₂ are independently selected from the group consisting of hydrogen, straight chain or branched alkyl groups having 1 to 3 carbon atoms selected from the group consisting of methyl, ethyl, propyl and isopropyl;
Y is CH or N;
X is selected from S, O, CH—NO₂, and N—CN;
A is hydrogen, unsubstituted or substituted lower (C₁₋₁₀)alkyl, said substituents being halogen (fluorine, chlorine, bromine, iodine), hydroxy, lower (C₁₋₄)alkoxy, lower (C₁₋₄)perhaloalkyl, lower (C₁₋₄)perhaloalkoxy; optionally substituted naphthyl; unsubstituted or substituted aromatic or non aromatic 5-6 membered rings with or without one to four heteroatoms independently selected from the group consisting of oxygen, nitrogen and sulphur, said substituents independently selected from one or more groups such as halogen (fluorine, chlorine, bromine, iodine), nitro, cyano, hydroxy, lower (C₁₋₄) alkyl, lower(C₁₋₄)alkoxy, lower (C₁₋₄)perhaloalkyl, lower (C₁₋₄)perhaloalkoxy, BR₃, substituted or unsubstituted five or six membered heterocyclic ring systems containing one to four heteroatoms selected from the group consisting of oxygen, nitrogen and sulphur, said heterocycylic substituents being (C₁-C₈)alkanoyl, lower (C₁-C₄)alkyl, lower (C₁-C₄)alkoxy carbonyl, N lower (C₁-C₄)alkylaminocarbonyl, N,N-dilower(C₁-C₄)alkylaminocarbonyl, N-lower (C₁-C₄)alkylaminothiocarbonyl, N,N-di(lower alkyl)(C₁-C₄)aminothiocarbonyl, N-lower (C₁-C₄)alkyl sulphonyl, phenyl substituted lower (C₁-C₄)alkyl sulphonyl, N-lower (C₁-C₄)alkyl amino, N,N-di(lower alkyl)(C₁-C₄)amino, unsubstituted or substituted phenyl, said substituents being halogen (fluorine, chlorine, bromine, iodine), hydroxy, lower (C₁₋₄)alkoxy, lower (C₁₋₄)perhaloalkyl, lower (C₁₋₄)perhaloalkoxy, nitro, cyano, amino, N(R₄)₂, 5-6 membered heterocyclic rings, the preferred heterocycles being 1,3-imidazolyl; 1,2,4 triazolyl; —CHR₅R₆;
wherein
R₃ is five or six membered aromatic or non aromatic ring with or without heteroatoms selected from the group consisting of oxygen, nitrogen and sulphur;
B is independently selected from (CH₂)ₘ, —S, —O(CH₂)ₘ, —S(CH₂)ₘ;
m is an integer from 1 to 4;
R₄ is hydrogen, unsubstituted or substituted lower (C₁₋₄)alkyl;
R₅ is —COQ, where Q=OR₄, —N(R₄)₂; and
R₆ is independently selected from hydrogen, straight chain or branched alkyl with or without substituents, the said substituents being halogen (e.g. fluorine, chlorine, bromine or iodine), hydroxy, lower (C₁₋₄)alkyl, lower (C₁₋₄)alkoxy, lower (C₁₋₄)perhaloalkyl, lower (C₁₋₄)perhaloalkoxy, SR₄; phenyl or phenyl substituted with halogen (fluorine, chlorine, bromine, iodine), hydroxy, lower (C₁₋₄)alkoxy, lower (C₁₋₄)perhaloalkyl, lower (C₁₋₄)perhaloalkoxy, SR₄; heterocyclic rings or substituted heterocyclic rings with heteroatoms selected from oxygen, nitrogen and sulphur, substituents on heterocyclic rings are independently selected from halogen (fluorine, chlorine, bromine, iodine), hydroxy, lower (C₁₋₄)alkyl, lower (C₁₋₄)alkoxy, lower (C₁₋₄)perhaloalkyl, lower (C₁₋₄)perhaloalkoxy, SR₄; phenyl or phenyl substituted with halogen (e.g. fluorine, chlorine, bromine or iodine), hydroxy, lower (C₁₋₄)alkoxy, lower (C₁₋₄)perhaloalkyl, lower (C₁₋₄)perhaloalkoxy or SR₄; the preferred heterocyclic rings are imidazole and indole.
